(19) United States
(12) Patent Application Publication
(10) Pub. No.: US 2023/0272464 A1
(43) Pub. Date: Aug. 31, 2023

(54) MODIFIED NUCLEOTIDES AND METHODS FOR DNA AND RNA POLYMERIZATION AND SEQUENCING

(71) Applicant: SeNA Research, Incorporated, Marietta, GA (US)

(72) Inventor: Zhen Huang, Marietta, GA (US)

(21) Appl. No.: 17/502,393

(22) Filed: Oct. 15, 2021

Related U.S. Application Data
(63) Continuation of application No. PCT/US2020/028110, filed on Apr. 14, 2020.
(60) Provisional application No. 62/835,240, filed on Apr. 17, 2019.

(51) Int. Cl. C12Q 1/6848

(52) U.S. Cl. CPC C12Q 1/6848; C12Q 1/6869

(57) ABSTRACT

Modified nucleotides, such as α-phosphoseleno-nucleotides (dNTPαSe and NTPαSe), can be incorporated into nucleic acids by enzymatic processes in a similar manner as naturally occurring nucleotides. Altering the properties of modified nucleotides can alter the interaction between the nucleotide and the enzyme. Enzymatic incorporation of modified nucleotides may occur at a lower rate than for native nucleotides, and can significantly inhibit misincorporation of nucleotides into nucleic acids during enzymatic extension and/or polymerization processes.

### CROSS REFERENCE TO RELATED APPLICATIONS

This application is a continuation of PCT International Patent Application No. PCT/US2020/028110, filed on 14 Apr. 2020, which claims priority to U.S. Provisional Pat. Application No. 62/835,240, filed Apr. 17, 2019, the disclosures of which are hereby incorporated by reference herein in their entirety.

### BACKGROUND

Nucleic acid polymerization plays a major role in biology and living systems, including DNA replication, RNA transcription, reverse transcription and genetic information storage. Biotechnology and disease diagnostics also rely on DNA and RNA synthesis with high fidelity and specificity.

### SUMMARY

This summary is provided to introduce a selection of concepts in a simplified form that are further described below in the detailed description. This summary is not intended to identify required or essential features of the claimed subject matter. Nor is this summary intended to be used to limit the scope of the claimed subject matter.

Certain aspects disclosed herein are directed to DNA polymerization, and the incorporation of different functionalities into DNA, such as modified nucleotides with derivatized nucleobases, sugars and phosphates disclosed herein. In certain aspects disclosed herein, DNA polymerase recognition of these selenium-modified dNTPs can lead to enzymatic extension and/or polymerization reactions with improved specificity. In certain aspects disclosed herein, reverse transcriptase recognition of these selenium-modified dNTPs can lead to enzymatic reactions with improved specificity. Certain aspects disclosed herein are directed to RNA polymerization, and the incorporation of different functionalities into RNA, such as modified nucleotides with derivatized nucleobases, sugars and phosphates disclosed herein. In certain aspects disclosed herein, RNA polymerase recognition of these selenium-modified NTPs can lead to enzymatic reactions with improved specificity.

In some aspects, the invention disclosed herein is directed to an enzymatic process for forming a nucleic acid product mixture. Such processes can comprise annealing a primer or promoter sequence to a template sequence, and extending the primer sequence or synthesizing the nucleic acid product in the presence of an extension or polymerase enzyme and a nucleotide mixture comprising at least one modified nucleotide to form a modified nucleic acid. In certain aspects, an amount of nonspecific nucleic acid products in the product mixture can be less than that of an otherwise identical process using an analogous native nucleotide.

In other aspects, the invention disclosed herein is directed to a reagent mixture for conducting nucleic acid extension and/or polymerization reactions, the mixture comprising a DNA or RNA primer sequence, a DNA template sequence, a DNA polymerase enzyme, and a nucleotide mixture. In certain aspects, the nucleotide mixture can comprise Se-modified nucleotide(s) and/or S-modified nucleotide(s) selected from the group consisting of dATPαSe, dCTPαSe, dGTPαSe, TTPαSe (or dTTPαSe), dUTPαSe, 2-Se-TTP (or 2-Se-dTTP), 2-Se-dUTP, 2-Se-TTPαSe (or 2-Se-dTTPαSe), 2-Se-dUTPαSe, dATPαS, dCTPαS, dGTPαS, TTPαS (or dTTPαS), dUTPαS, 2-S-TTP (or 2-S-dTTP), 2-S-dUTP, 2-S-TTPαS (or 2-S-dTTPαS) and 2-S-dUTPαS, and non-analogous native nucleotide(s) selected from the group consisting of dATP, dCTP, dGTP, TTP (or dTTP) and dUTP. In other aspects, the invention disclosed herein is directed to a reagent mixture for conducting nucleic acid synthesis reactions, the mixture comprising a DNA promoter sequence, a DNA template sequence, a RNA polymerase enzyme, and a nucleotide mixture. In certain aspects, the nucleotide mixture can comprise Se-modified nucleotide(s) and/or S-modified nucleotide(s) selected from the group consisting of ATPαSe, CTPαSe, GTPαSe, UTPαSe, rTTPαSe, 2-Se-UTP, 2-Se-rTTP, 2-Se-UTPαSe, 2-Se-rTTPαSe, ATPαS, CTPαS, GTPαS, UTPαS, rTTPαS, 2-S-UTP, 2-S-rTTP, 2-S-UTPαS and 2-S-rTTPαS, and non-analogous native nucleotide(s) selected from the group consisting of ATP, CTP, GTP, UTP and rTTP. In other aspects, the invention disclosed herein is directed to a reagent mixture for conducting nucleic acid extension and/or synthesis reactions, the mixture comprising a DNA or RNA primer sequence, a RNA template sequence, a reverse transcriptase enzyme, and a nucleotide mixture. In certain aspects, the nucleotide mixture can comprise Se-modified nucleotide(s) and/or S-modified nucleotide(s) selected from the group consisting of dATPαSe, dCTPαSe, dGTPαSe,TTPαSe (or dTTPαSe), dUTPαSe, 2-Se-TTP (or 2-Se-dTTP), 2-Se-dUTP, 2-Se-TTPαSe (or 2-Se-dTTPαSe), 2-Se-dUTPαSe, dATPαS, dCTPαS, dGTPαS, TTPαS (or dTTPαS), dUTPαS, 2-S-TTP (or 2-S-dTTP), 2-S-dUTP, 2-S-TTPαS (or 2-S-dTTPαS) and 2-S-dUTPαS, and non-analogous native nucleotide(s) selected from the group consisting of dATP, dCTP, dGTP, TTP (or dTTP) and dUTP. The following 2-Se-pyrimidine triphosphates and 2-S-pyrimidine triphosphates represent non-limiting embodiments of the modified nucleotides disclosed herein:

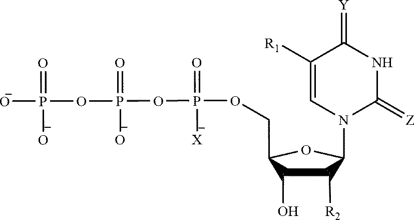

Other aspects of the invention disclosed herein are directed to selenium- or sulfur-modified nucleotide selected from the group consisting of 3′-O-N₃-dATPαSe, 3′-O-N₃-dCTPαSe, 3′-O-N₃-dGTPαSe,3′-O-N₃-dTTPαSe, ddCTPαSe-N₃-Bodipy-FL-510, ddUTPαSe-N₃-R6G, ddATPαSe-N₃-ROX, ddGTPαSe-N₃-Cy5, 3′-O-N₃-dATPαS, 3′-O-N₃-dCTPαS, 3′-O-N₃-dGTPαS, 3′-O-N₃-dTTPαS, 3′-O-N₃-dUTPαS, ddCTPαS-N₃-Bodipy-FL-510, ddUTPαS-N₃-R6G, ddATPαS-N₃-ROX, ddGTPαS-N₃-Cy5. Reagent mixtures comprising Se-modified or S-modified sequencing nucleotides are also disclosed herein, and can comprise a primer sequence, a template sequence, a polymerase enzyme, any Se-modified or S-modified sequencing nucleotide disclosed herein, and non-analogous native nucleotides.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects and embodiments may be directed to various feature combinations and sub-combinations described in the detailed description.

### BRIEF DESCRIPTION OF THE FIGURES

FIG. 1 presents results from gel electrophoresis studies comparing the extension of primer sequences with isolated diastereomers of Se-modified nucleotide and (a) DNA polymerase I, (b) Klenow Fragment, or (c) Bst polymerase as the extension enzyme

FIG. 2 presents results from a gel electrophoresis experiment using diastereomeric mixtures of each Se-modified nucleotide in the presence of three other non-analogous native nucleotides and either Klenow fragment or Bst polymerase as the extension enzyme.

FIG. 3 presents results from gel electrophoresis studies to determine the extension and/or polymerization rate of enzymatic extension and polymerizations in the presence of an Se-modified nucleotide and three non-analogous native nucleotides, compared to an analogous extension using all native nucleotides.

FIG. 4 presents the results of a gel electrophoresis study as a color-reversed image comparing extension in the presence of dCTPαSe I to extension in the presence of native nucleotides.

FIG. 5A presents results of a gel electrophoresis experiment characterizing the suppression of spontaneous DNA polymerization.

FIG. 5B presents results of a gel electrophoresis experiment characterizing the suppression of spontaneous DNA polymerization (60 min) in the presence of 1-4 Se-modified nucleotides, in the presence of the primer only, or the template only.

FIG. 6 presents results of a gel electrophoresis experiment characterizing the suppression of non-specific DNA polymerization (90 min).

FIG. 7 presents graphs detailing results from comparative sequencing experiments.

FIGS. 8A-8B present overlapped mass spectra plots of Se-modified DNA treated with or without hydrogen peroxide, either at room temperature (FIG. 8A) or at 50° C. (FIG. 8B).

FIG. 9 presents results from gel electrophoresis studies to determine the extension and/or polymerization rate of enzymatic extension and polymerizations in the presence of an S-modified nucleotide and three non-analogous native nucleotides, compared to an analogous extension and/or polymerization using all native nucleotides.

FIGS. 10A-10C present reagents (FIG. 10A) and results of a gel electrophoresis experiment characterizing the suppression of spontaneous DNA polymerization (FIG. 10B) and results of a gel electrophoresis experiment characterizing the suppression of spontaneous DNA polymerization (60 min) (FIG. 10C) in the presence of 1-4 S-modified nucleotides, in the presence of the primer only, or the template only.

FIGS. 11A-11B present reagents (FIG. 11A) and gel electrophoresis results for (FIG. 11B) polymerase reactions using dCTPαS and dTTPαS, compared to all native nucleotides.

FIGS. 12A-12D present presents gel electrophoresis results examining replication in the presence of native nucleotides vs. a combination of dCTPαS and other natives.

FIGS. 13A-13B present gel electrophoresis results on the amplification of various sequences using native dCTP vs. dCTPαS.

FIG. 14 presents HRMS characterization of 2-Se-dTTP.

FIG. 15 presents ¹H-NMR characterization of 2-Se-dTTP.

FIG. 16 presents ¹³C-NMR characterization of 2-Se-dTTP.

FIG. 17 presents ³¹P-NMR characterization of 2-Se-dTTP.

FIGS. 18A-18D, 19A-19D, 20A-20D, and 21 present gel electrophoresis studies comparing the specificity and fidelity of 2-Se-TTP to native nucleotides.

FIGS. 22A-22D, 23A-23C, 24A-24D, and 25 present gel electrophoresis studies comparing the specificity and fidelity of 2-S-TTP to native nucleotides.

### DEFINTIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2^{nd} Ed (1997), can be applied, as long as that definition does not conflict with any other disclosure or definition applied herein, or render indefinite or non-enabled any claim to which that definition can be applied. To the extent that any definition or usage provided by any document incorporated herein by reference conflicts with the definition or usage provided herein, the definition or usage provided herein controls.

Herein, features of the subject matter can be described such that, within particular aspects and/or embodiments, a combination of different features can be envisioned. For each and every aspect, and/or embodiment, and/or feature disclosed herein, all combinations that do not detrimentally affect the designs, processes, and/or methods described herein are contemplated with or without explicit description of the particular combination. Additionally, unless explicitly recited otherwise, any aspect, and/or embodiment, and/or feature disclosed herein can be combined to describe inventive features consistent with the present disclosure.

Regarding claim transitional terms or phrases, the transitional term “comprising,” which is synonymous with “including,” “containing,” “having,” or “characterized by,” is open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristics of the claimed invention. A “consisting essentially of” claim occupies a middle ground between closed claims that are written in a “consisting of” format and fully open claims that are drafted in a “comprising” format. Absent an indication to the contrary, describing a composition or method as “consisting essentially of” is not to be construed as “comprising,” but is intended to describe the recited element that includes materials or steps which do not significantly alter the composition or method to which the term is applied. For example, a nucleotide mixture consisting essentially of a Se-modified nucleotide can include impurities typically present in a commercially produced or commercially available sample of the Se-modified nucleotide. When a claim includes different features and/or feature classes (for example, a process step, reagent process features, and/or reagent stream features, among other possibilities), the transitional terms comprising, consisting essentially of, and consisting of apply only to the feature class to which it is utilized, and it is possible to have different transitional terms or phrases utilized with different features within a claim. For example, a process can comprise several recited steps (and other non-recited steps), but utilize a reagent mixture consisting of specific components; alternatively, consisting essentially of specific components; or alternatively, comprising the specific components and other non-recited components. While compositions and processes are described in terms of “comprising” various components or steps, the compositions and methods can also “consist essentially of” or “consist of” the various components or steps, unless specifically stated otherwise. For example, a nucleotide mixture consistent with certain embodiments of the present invention can comprise; alternatively, consist essentially of; or alternatively, consist of; a Se-modified nucleotide.

The terms “a,” “an,” and “the” are intended to include plural alternatives, e.g., at least one, unless otherwise specified. For instance, the disclosure of “a Se-modified nucleotide” is meant to encompass one, or mixtures or combinations of more than one, Se-modified nucleotide, unless otherwise specified.

For any particular compound or group disclosed herein, any name or structure presented is intended to encompass all conformational isomers, regioisomers, and stereoisomers that can arise from a particular set of substituents, unless otherwise specified. For example, a general reference to α-P-seleno-deoxyadenosinetriphosphate (ATPαSe) includes both the Rp and Sp diastereomers of the selenium modified nucleotide. The name or structure also encompasses all enantiomers, diastereomers, and other optical isomers whether in enantiomeric or racemic forms, as well as mixtures of stereoisomers, as would be recognized by a skilled artisan, unless otherwise specified.

As used herein, the term “native nucleotide” refers to a nucleotide that is analogous to a related modified nucleotide except for the specific modification of the modified nucleotide. Thus, in certain aspects, each modified nucleotide can have an analogous native nucleotide, and vice versa. Moreover, a modified nucleotide may have any number of non-analogous native nucleotides where the specific modification present in the modified nucleotide is not present in a complementary nucleotide. For instance, aspects comprising a modified nucleotide of dATPαSe can comprise dATP as an analogous native nucleotide lacking the α-phosphoseleno modification, and also may comprise dCTP, dGTP, and/or dTTP as non-analogous native nucleotides.

Similarly, the term “native nucleic acid” refers to a nucleic acid that is identical to a related modified nucleic acid except for the specific modification present in a modified nucleic acid. In certain aspects, native nucleic acids can be entirely comprised of native nucleotides. In certain aspects, a native nucleotide may refer to a naturally occurring nucleotide such as dATP, dCTP, and the like. Alternatively, a native nucleotide may refer to a synthetic nucleotide. In either case, the term native nucleotide is meant to represent an analog to the modified nucleotide prior to, or lacking the specific modification in the modified nucleotide to which it refers. Thus, in this sense, each native nucleotide disclosed herein can be related to an analogous modified nucleotide by a particular modification, and not restricted to any particular nucleotide, naturally-occurring or otherwise. Thus, native nucleotides may refer to nucleotides having modifications to the base, sugar, or phosphate chain of the nucleotide. It follows that “modified nucleotides” may also be defined herein relative to a native nucleotide base state. For any aspects herein where the relationship between modified nucleotide and its native nucleotide may not be explicit, the modified nucleotide can generally encompass any modifications disclosed herein. For instance, in certain aspects the modified nucleotide can differ from its analogous native nucleotide by the presence of a selenium atom at the α-phosphate group as opposed to the native nucleotide having an oxygen in the same position.

Further, the term “naturally-occurring nucleotide” refers to nucleotides having chemical structures identical to those commonly found in nature (e.g., DNA and RNA nucleotides) and is not restricted to any particular source of the nucleotide. For instance, naturally-occurring nucleotides as contemplated herein may be isolated from a natural source, or alternatively may be synthesized by common chemical procedures, where convenient. In this manner, references to naturally occurring nucleotides herein refer to the chemical structure of the nucleotide, and not its source or chemical preparation.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

Although any methods and materials similar or equivalent to those described herein can be used in the practice or testing of the invention, the typical methods and materials are herein described.

All publications and patents mentioned herein are incorporated herein by reference. The publications and patents mentioned herein can be utilized for the purpose of describing and disclosing, for example, the constructs and methodologies that are described in the publications, which might be used in connection with the presently described invention. The publications discussed throughout the text are provided solely for their disclosure prior to the filing date of the present application. Nothing herein is to be construed as an admission that the inventors are not entitled to antedate such disclosure by virtue of prior invention.

Applicants reserve the right to proviso out or exclude any individual members of any such group, including any sub-ranges or combinations of sub-ranges within the group, that can be claimed according to a range or in any similar manner, if for any reason Applicants choose to claim less than the full measure of the disclosure, for example, to account for a reference that Applicants may be unaware of at the time of the filing of the application. Further, Applicants reserve the right to proviso out or exclude any individual substituents, analogs, compounds, ligands, structures, or groups thereof, or any members of a claimed group, if for any reason Applicants choose to claim less than the full measure of the disclosure, for example, to account for a reference that Applicants may be unaware of at the time of the filing of the application.

### DETAILED DESCRIPTION

The invention disclosed herein is directed generally to processes for the enzymatic extension and/or synthesis of nucleic acid sequences. Such processes can incorporate the modified nucleotides to form nucleic acid sequences with improved product quality, fidelity and specificity. Reagent mixtures comprising modified nucleotides are also contemplated herein. Certain aspects are directed to modified sequencing nucleotides.

### Modified Nucleotides

Generally, the modified nucleotides disclosed herein are modified with reference to an analogous native nucleotide. In certain aspects, the modified nucleotides may differ from the analogous native nucleotide by substitution of one or more atoms of the native nucleotide with an atom having a larger atomic radius. In some aspects, the substitute atom can be within the same Periodic Group as the atom of the native nucleotide. For instance, an oxygen atom of a naturally-occurring nucleotide can be replaced with sulfur, or alternatively selenium. Alternatively, a carbon atom of the naturally-occurring nucleotide may be substituted with a silicon atom, or a nitrogen atom of the naturally-occurring nucleotide may be substituted by a phosphorus atom. Modified nucleotides having multiple substitutions of atoms are also contemplated herein.

Native nucleotides subject to the modifications disclosed herein can be either a naturally-occurring nucleotide or a derivatized nucleotide, and can be produced or isolated from either synthetic or natural sources. For instance, native nucleotides contemplated herein can include DNA nucleotides (dATP, dGTP, dCTP, TTP (or dTTP) and dUTP) and RNA nucleotides (ATP, GTP, CTP, UTP and rTTP). Each of these naturally occurring nucleotides may be modified at any position, where the modification is effective to improve the enzymatic extension and/or polymerization processes disclosed below.

Modified nucleotides contemplated herein can be modified at any position compared to an analogous native nucleotide, including within a phosphate group, the sugar, the base, or any combination thereof. Thus, in certain aspects, the modified nucleotide can be dATPαS, dCTPαS, dGTPαS, TTPαS (or dTTPαS), dUTPαS, 2-S-TTP (or 2-S-dTTP), 2-S-dUTP, 2-S-TTPαS (or 2-S-dTTPαS) and 2-S-dUTPαS, ATPαS, CTPαS, GTPαS, UTPαS, rTTPαS, 2-S-UTP and 2-S-rTTP, 2-S-UTPαS and 2-S-rTTPαS. Alternatively, the modified nucleotide can comprise an α-phosphoseleno group. In such aspects, the modified nucleotide can be dATPαSe, dCTPαSe,dGTPαSe,TTPαSe (or dTTPαSe), dUTPαSe, 2-Se-TTP (or 2-Se-dTTP), 2-Se-dUTP, 2-Se-TTPαSe (or 2-Se-dTTPαSe), 2-Se-dUTPαSe, ATPαSe, CTPαSe, GTPαSe, UTPαSe, rTTPαSe, 2-Se-UTP, 2-Se-rTTP, 2-Se-UTPαSe, 2-Se-rTTPαSe. Modifications of a native nucleotide at its sugar ring are also contemplated herein, and thus modified nucleotides of this disclosure can include 2′-S-ATP, 2′-S-CTP, 2′-S-GTP, 2′-S-TTP, 2′-S-dUTP, 2′-Se-ATP, 2′-Se-CTP, 2′-Se-GTP, 2′-Se-TTP and 2′-Se-dUTP. Modified nucleotides comprising modifications to the phosphate, sugar ring, and/or base as disclosed within U.S. Pat. Nos. 7,592,446, 7,982,030, and 8,354,524 are also contemplated herein, each of which is incorporated herein by reference in its entirety. Alternatively, modified nucleotides contemplated herein can include substitutions of any phosphorus atom in the phosphate group for a silicon atom.

Alternatively, or additionally, the modified nucleotide may comprise a modification to the base of an analogous native nucleotide. Thus, in aspects where the native nucleotide is a naturally-occurring DNA or RNA nucleotide, the modified nucleotide can be modified to include a sulfur or selenium atom at the 2-position of a thymine, uracil, or cytosine base, as in 2-S-dCTP, 2-S-CTP, 2-S-dUTP, 2-S-UTP, 2-S-TTP, 2-S-rTTP, 2-Se-dCTP, 2-Se-CTP, 2-Se-dUTP, 2-Se-UTP, 2-Se-TTP, or 2-Se-rTTP. Alternatively, or additionally, the thymine or uracil base can be modified at the 4-position, as shown in the Examples below.

Modified nucleotides having additional or alternative substitutions of heteroatoms at the nucleotide base are also contemplated herein. In still further aspects, modified nucleotides can comprise substitutions of atoms on non-naturally occurring nucleotides. In such aspects, the native nucleotide may be the same or different from a naturally occurring nucleotide at any combination of the phosphate, sugar ring, or base. For instance, sequencing nucleotides often can have a modified base structure to incorporate an optically active chemical moiety (e.g., a fluorescent group). For instance, sequencing nucleotides often can have a modified gamma-phosphate or gamma-phosphate structure for cleaving and offering a signal as an optically active chemical moiety (e.g., a fluorescent group). For instance, sequencing nucleotides often can have a modified sugar structure to incorporate, at each cycle of extension, one nucleotide with a chemically protecting moiety (e.g., a protecting 3′-CH₂-N₃ group) to allow single nucleotide incorporation at each cycle of extension. Optically active chemical moieties often can be incorporated into the sequencing nucleotide by direct modification to the base structure, or more commonly, by a linking group between the base and the optically active moiety. In certain aspects, the sequencing nucleotides may incorporate an optically active moiety with or without disrupting the ability of a polymerase enzyme to incorporate the sequencing nucleotide in a nucleic acid sequence during an enzymatic extension and/or synthesis of the nucleic acid. Modified sequencing nucleotides contemplated herein may be advantageously modified at the phosphate group, to preserve the structure of the optical moiety, linking group and base of the native sequencing model, while achieving the features of the processes described herein.

Thus, modified sequencing nucleotides contemplated herein can comprise a substitution of a heteroatom of the α-phospho group. In certain aspects, the modified sequencing nucleotide can comprise an α-phosphothio modification, an α-phosphoseleno modification, or combinations thereof. Thus, modified sequencing compounds contemplated herein can be selected from any of 3′-O-N₃-dATPαSe, 3′-O-N₃-dCTPαSe, 3′-O-N₃-dGTPαSe, 3′-O-N₃-dTTP, ddCTPαSe-N₃-Bodipy-FL-510, ddUTPαSe-N₃-R6G, ddATPαSe-N₃-ROX, ddGTPαSe-N₃-Cy5, 3′-O-N₃-dATPαS, 3′-O-N₃-dCTPαS, 3′-O-N₃-dGTPαS, 3′-O-N₃-dTTPαS, ddCTPαS-N₃-Bodipy-FL-510, ddUTPαS-N₃-R6G, ddATPαS-N₃-ROX, ddGTPαS-N₃-Cy5, or combinations thereof, as represented by the following structures.

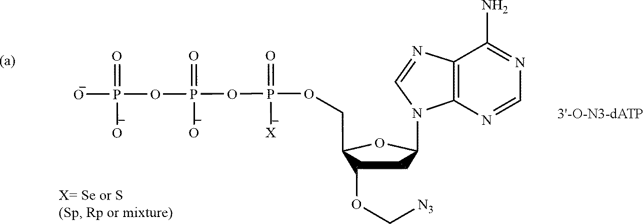

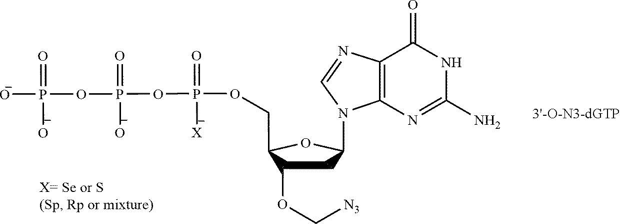

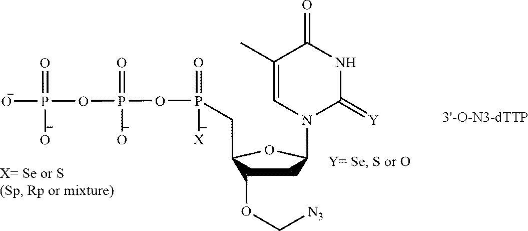

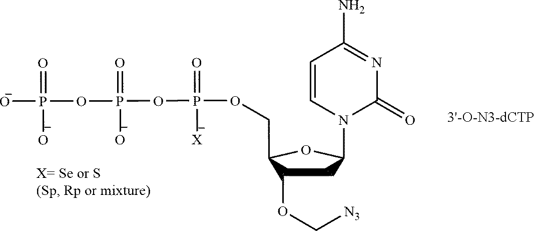

### Processes for Enzymatic Extension and Synthesis of Nucleic Acids

Generally, the processes disclosed herein may use any of the modified nucleotides described above (independently, or as part of reagent mixtures described below) to enzymatically extend and/or synthesize nucleic acid sequences. Such enzymatic extension and polymerization processes are not limited to any particular process or function, and generally can be any process that incorporates a nucleotide within a partial nucleotide sequence to extend the sequence and/or synthesize a nucleic acid. In certain aspects, the enzymatic process contemplated herein can be a cDNA synthesis, a PCR amplification, an isothermal amplification, or a nucleic acid sequencing process.

In certain aspects, the processes disclosed herein can comprise an annealing step to allow a primer or promoter nucleic acid sequence to bind to a template sequence, followed by an extending and/or synthesizing step to extend the primer sequence and/or synthesize nucleic acid to incorporate any modified nucleotide disclosed herein within to form a modified nucleic acid. In certain aspects, the template sequence can be the sequence targeted for replication, amplification, sequencing, etc., and the primer sequence can be a small fragment of a complementary nucleic acid sequence able to bind the template sequence and allow an extension and/or synthesis enzyme to extend the primer sequence or synthesize a nucleic acid. In other aspects, the process can further comprise a denaturation step to denature the modified nucleic acid and allow additional annealing and extending steps. In such embodiments, any number of cycles suitable to serve the purpose of the particular process is contemplated herein. Certain processes contemplated herein may have a number of cycles in a range from about 2 to about 100, from about 15 to about 75, from about 20 to about 60, or from about 20 to about 40. Similarly, processes contemplated herein may comprise at least 3 cycles, at least 5 cycles, at least 10 cycles, or at least 20 cycles.

Conditions of each step of the processes are also not limited to any particular temperature, pressure, solvent, reaction time, etc. and generally can be conducted under any conditions suitable for the particular processes. Moreover, the processes can be conducted in the presence of any reagent mixture, nucleotides, polymerization enzymes, or combinations thereof described herein or that may be suitable to complete the process. For instance, in certain aspects, the annealing and extending or synthesizing steps independently can be conducted in any reagent mixture disclosed herein suitable for the conditions of the process. The reagent mixture can be the same or different in any of the steps of the process.

Similarly, the temperature of any step can be any that are suitable to conduct the particular step or the process as a whole. In certain aspects, a temperature of the annealing step can be in a range from about 10° C. to about 60° C., from about 20° C. to about 50° C., or from about 25° C. to about 40° C. Likewise, the temperature of the extending step can be in any suitable range, and in a range from about 20° C. to about 90° C., from about 30° C. to about 70° C., or from about 40° C. to about 60° C. Denaturation steps may be conducted at somewhat higher temperatures to ensure the binding interactions between complementary strands are completely dissociated, for instance in a range from about 40° C. to about 100° C., or from about 60° C. to about 90° C.

In certain aspects, an amount of error-free modified nucleic acid present in the crude product mixture can be higher than that for an analogous process using only native nucleotides. For instance, an amount of misincorporation of the modified nucleotide during the extending or synthesizing step can be less than that of an otherwise identical process using a native nucleotide. While not being bound by theory, it is believed that modifications to nucleotides through substitution of an atom in the native nucleotide with an atom having a larger atomic radius (e.g., substituting oxygen with selenium) may improve the fidelity of the extension and polymerization enzyme by reducing the amount of nucleotides that are misincorporated into nucleic acid during the extending or synthesizing step. In certain aspects, an error rate of the extension and/or polymerization enzyme can be less than about 1 per 10⁵ base pairs, less than about 1 per 10⁶ base pairs, less than about 1 per 10⁷ base pairs, or less than about 1 per 10⁸ base pairs.

Thus, the amount of modified nucleic acids in the product mixture that are not complementary to the template sequence can be less than about 10%, less than about 5%, less than about 3%, less than about 2%, less than about 1%, less than about 0.5%, or less than about 0.1%, due to the increased fidelity of the processes disclosed herein. As any such non-complementary sequences can have similar molecular weights, separation of such sequences from the product mixture can be impractical. Thus, improving the fidelity by processes disclosed herein may also improve the purity of any subsequent isolated product.

Inserting a phosphorothioate linkage near the 3′ end of primer can inhibit mis-priming between primer with primer or template, so it has been used in DNA amplification (such as PCR), for higher specificity and less nonspecific product. Even so, the application of phosphorothioated primer remains hindered by insufficient effect on nonspecific amplification and inconvenient preparation of diastereomerically-pure oligonucleotides. Surprisingly, the processes herein can demonstrate an amount of nonspecific byproducts in the product mixture due to mispriming of the primer sequence during the annealing step also may be reduced in the presence of modified nucleotides and reagent mixtures disclosed herein. Thus, an amount of the modified nucleic acid in the product mixture, relative to non-specific products (and prior to isolation) can be at least about 90%, at least about 95%, at least about 98 wt. %, at least about 99 wt. %, or at least about 99.5 wt. %. In other aspects, the amount of modified nucleic acid in the product mixture, relative to non-specific products, can be in a range from about 80 wt. % to about 99.99 wt. %, from about 90 wt. % to about 99 wt. %, or from about 95 wt. % to about 99 wt. %. Certain aspects may further comprise isolating the modified nucleic acid from the product mixture to form a purified modified nucleic acid. In certain aspects, the isolating step can include gel electrophoresis, or any other suitable method to isolate the target product from the product mixture.

In certain aspects, an extension and/or polymerization rate of the extension and/or polymerization enzyme with the modified nucleotide can be lower than that for the native nucleotide. In certain aspects, the extension and/or polymerization rate can be in any range disclosed herein (e.g., from about 1 base pairs/second to about 10,000 base pairs per second, from about 1000 to about 8000 base pairs per second, from about 2000 to about 6000 base pairs per second, from about 3000 to about 5000 base pairs per second). While not being bound by theory, the substitution of an atom having a larger atomic radius may contribute to the observed reduction in extension and/or polymerization rate, and increases in fidelity and specificity. Surprisingly, processes that include α-phosphoseleno modified nucleotides may be particularly effective in reducing the extension and/or polymerization rate, without prolonging the time period necessary to complete extending or synthesizing steps of the process. Thus, in certain aspects the extension and/or polymerization rate of the extension and/or polymerization enzyme for the modified nucleotide can be any amount or percentage less than that relative to an extension and/or polymerization rate of the extension and/or polymerization enzyme for an analogous native nucleotide (e.g., about 90 %, about 80%, about 60%, about 50%, about 30%, about 20%, about 10% or about 1% less, or at least about 1 base pairs per second, at least about 500 base pairs per second, or at least about 1000 base pairs per second less).

Processes disclosed herein may further comprise an oxidizing or hydrolyzing step to convert the modified nucleic acid to the native nucleic acid. Oxidizing step may be conducted in the presence of an oxidant, and under any conditions suitable for the oxidation, for instance, within any reagent mixture or product mixture disclosed herein without further isolation. Oxidants that may be suitable can include hydrogen peroxide solutions (e.g. 3% H₂O₂). Moreover, the temperature of the oxidizing step is not particularly limited, and in certain aspects can be in a range from about 0 to about 100° C. Alternatively, the oxidation temperature can be in a range that allows the modified nucleic acid to be stable at room temperature, while being oxidized at relatively mild temperatures, for instance in a range from about 40° C. to 80° C., or from about 40° C. to 60° C.

### Reagent Mixtures

Reagent mixtures disclosed herein generally are suitable for any of the processes described above, and can incorporate any of the modified nucleotides (and corresponding analogous and non-analogous native nucleotides) described above. In some aspects, reagent mixtures disclosed herein can comprise a primer or promoter sequence, a template sequence, an extension and/or polymerization enzyme, and a nucleotide mixture. However, the reagent mixture may also further comprise any number of additional elements that may facilitate the processes described above. For instance, the reagent mixture can comprise any number of diluents and/or buffers to facilitate the reaction. In certain aspects, the reagent mixture can comprise polar solvents such as water, alcohols, or both. Additionally, the reagent mixture also may comprise nonpolar solvents to facilitate a denaturing step. The reagent mixture also can comprise salts in any concentration suitable for any process disclosed herein.

Any concentration of the primer, promoter and template sequences may be suitable for the processes disclosed herein; however, in some aspects the primer or promoter sequence and template sequence independently can have a concentration of from about 1 yoctoM to about 1 mM. The primer sequence may consist of naturally-occurring nucleotides, or may comprise any amount of modified nucleotides described herein.

In certain processes, the length of the primer sequence may affect the amount of mispriming, as longer sequences may anneal to themselves during the annealing step leading to relatively short and non-complementary extended sequences. In contrast, shorter primer sequences may result in non-specific binding to a complementary sequence of the template strand, and also result in relatively short sequences. Thus, the length of the primer sequence in the reagent mixture may be process-dependent. In some aspects, the length of the primer sequence can be in a range from about 3 to about 100 bases, from about 10 to about 50 bases, or from about 10 to about 30 bases. Similarly, the length of the template sequence is not limited to any particular length, and can be any length generally suitable for the processes described herein. Thus, the length of the primer sequence in the reagent mixture may be process-dependent. In some aspects, the length of the primer sequence can be in a range from about 50 to about 10,000 bases, from about 100 to about 5,000 bases, or from about 100 to about 3,000 bases.

Generally, the reagent mixture can comprise any number or combination of modified and native nucleotides described above, such as may be suitable to extend a primer sequence to the length of a template sequence, or facilitate any process disclosed herein. In certain aspects, the reagent mixture can comprise a mixture of naturally-occurring (native) nucleotides, and any relative or absolute amount of analogous modified nucleotides. The reagent mixture can comprise a single modified nucleotide, with or without an analogous native nucleotide. In some aspects, the reagent mixture also can comprise any number of non-analogous native nucleotides (e.g., one, two, three, four, five, etc.), and in any nucleotide concentration disclosed herein. In mixtures comprising an analogous native nucleotide, the molar ratio of modified nucleotide analogous native nucleotide is not limited to any particular amount, and may be any minimal amount suitable to facilitate the processes described herein. For instance the molar ratio of modified nucleotide:analogous native nucleotide can be in a range from 1:100 to 10:1, from 1:10 to 10:1, from 1:5 to 5:1, or from 1:2 to 10:1, or analogous native nucleotide may not even be included in the reagent mixture. In other aspects, more than one modified nucleotide can be present in any amount or ratio relative to the analogous native nucleotide disclosed herein (e.g., in the absence of the analogous native nucleotide, in a molar ratio from 1:100 to 10:1, etc.), or analogous native nucleotide may not even be included in the reagent mixture.

In certain aspects the reagent mixture can comprise a single modified nucleotide and three non-analogous native nucleotides. Alternatively, the reagent mixture can comprise two modified nucleotides and two non-analogous native nucleotides. In other aspects, the reagent mixture can comprise three modified nucleotides and one non-analogous native nucleotides. In still other aspects, the reagent mixture can comprise four modified nucleotides in the absence of a native nucleotide. In other aspects, the reagent mixture can comprise four naturally-occurring nucleotides and any number of modified nucleotides (e.g., 1, 2, 3, 4, etc.), each in any concentration or molar ratio disclosed herein.

Concentrations of nucleotides in the reagents mixtures disclosed herein are not limited to any particular amount, and can be in any amount suitable for processes disclosed herein. In certain aspects, each nucleotide (e.g., modified, naturally-occurring, native, sequencing, etc.) can have a concentration in any reagent mixture disclosed herein in a range from about 1 nM to about 100 mM, from about 10 nM to about 100 mM, from about 10 nM to about 10 mM, from about 1 µM to about 500 µM, or from about 50 µM to about 300 µM.

Nucleotide concentrations suitable for reagent mixtures and processes disclosed herein can depend, at least in part, on the nature of the extension and/or polymerization enzyme. As stated above, the extension and/or polymerization enzyme is not limited to any particular enzyme, and may be any that is capable of extending a primer sequence annealed to a template strand during the extending step of any process disclosed herein. For instance, in certain aspects the extension and/or polymerization enzyme may be a mammalian (e.g., human) enzyme, a bacterial enzyme, or fragments and combinations thereof. In certain aspects, the extension and/or polymerization enzyme can comprise a DNA polymerase, a RNA polymerase, a reverse transcriptase, or fragments and/or combinations thereof. In other aspects, the extension and/or polymerization enzyme can be human DNA polymerase I, Klenow fragment, or Bst polymerase.

### EXAMPLES

The invention is further illustrated by the following examples, which are not to be construed in any way as imposing limitations to the scope of this invention. Various other aspects, embodiments, modifications, and equivalents thereof which, after reading the description herein, can suggest themselves to one of ordinary skill in the art without departing from the spirit of the present invention or the scope of the appended claims.

### Preparation Of Alpha-Phosphate Seleno Modified Nucleotides (dNTPαSe)

2′-deoxynucleoside 5′-(alpha-P-seleno)-triphosphate (dNTPαSe) was synthesized via the protection-free one-pot strategy reported in. The native nucleoside (1 mmol), tributylammonium pyrophosphate (945 mg, 2 mmol, 2 equivalents) and 3H-1, 2-benzothiaselenol-3-one (BTSe, 435 mg, 2 mmol, 2 equiv) were dried in separate flasks under high vacuum for 1 h. DMF (1.5 mL) and tributylamine (TBA, 3.0 mL) were added to the pyrophosphate as a solvent. Anhydrous 2-chloro-4H-1,3,2-benzodioxaphosphorin-4-one (405 mg, 2 mmol, 2 equiv) dissolved in DMF (3.0 mL) was then injected into the pyrophosphate solution. This reaction mixture was stirred under argon at room temperature for 60 min and then injected into the flask containing the dried native nucleoside dissolved (thymidine and deoxycytidine were dissolved in 1.5 mL DMF; deoxyadenosine was dissolved in a mixed solvent of 1.0 mL DMF and 1.5 mL DMSO, and deoxyguanosine was dissolved in 3.0 mL DMSO). 3H-1,2-benzothiaselenol-3-one (BTSe) dissolved in dioxane (2.5 mL) was then injected to the reaction mixture and stirred at room temperature for 1h. Water (approximately twice the volume of the reaction solution) was then added to the reaction mixture, and stirred at room temperature for 2 h. Impurities in the crude product mixture were removed with ethanol/NaCl precipitation in the presence of fresh DTT (2 mM).

Crude products were further purified by RP-HPLC over an Ultimate XB-C18 column (10 µm, 30×250 mm, form Welch, China). Samples were eluted (15 mL/min) with a linear gradient from 90% Buffer A (20 mm triethylammonium acetate (TEAAc), pH 6.6) and 10% Buffer B (50% acetonitrile in water, 20 mm TEAAc, pH 6.6) to 20% Buffer B over 50 min. The purified Se-modified nucleotide diastereomers were lyophilized and re-dissolved separately in a small amount of a solution of 10 mM tris(hydroxymethyl)aminomethane/HCl (Tris-HCl, pH 7.5) and 20 mM of DTT and stored at -80° C. The synthesized Se-modified nucleotides (6) were analyzed by RP-HPLC (FIG. 1), and the purified products were eluted (1 mL/min) with a linear gradient from 95% Buffer A and 5% Buffer B to 26% Buffer B at 21 min on Ultimate AQ-C18 column (5 µm, 4.6×250 mm, form Welch, China). Concentration and quantity of each Se-modified nucleotide were determined via UV analysis with MULTISKAN GO with µDrop Plate (Thermo Scientific), which indicated an overall yield greater than 40%.

All synthesized Se-modified nucleotides were analyzed by HR-MS, ¹H-NMR, ¹³C-NMR, and ³¹P-NMR, as shown in the tables below.

TABLE 1

¹H-NMR chemical shifts (δ, ppm) of dNTPαSe analogs in D₂O

| Compounds | H8 | H2 | H6 | H5 | H1′ | H2′ | H3′ | H4′ | H5′ |
|---|---|---|---|---|---|---|---|---|---|
| dATPαSeI | 8.64 | 8.26 | | | 6.53 | 2.86, 2.60 | 4.22 | 4.33 | 4.38-4.28 |
| dATPαSeII | 8.58 | 8.24 | | | 6.52 | 2.85, 2.61 | 4.24 | 4.30 | 4.27-4.19 |
| dGTPαSe I | 8.18 | | | | 6.26 | 2.79, 2.52 | 4.26 | 4.30 | 4.32-4.20 |
| dGTPαSe II | 8.08 | | | | 6.28 | 2.80, 2.52 | 4.26 | 4.30 | 4.32-4.20 |
| dCTPαSe I | | | 8.08 | 6.18 | 6.35 | 2.43, 2.34 | 4.25 | 4.30 | 4.66, 4.30 |
| dCTPαSe II | | | 8.05 | 6.07 | 6.34 | 2.42, 2.34 | 4.25 | 4.30 | 4.66, 4.30 |
| TTPαSe I | | | 7.79 | 1.98 | 6.37 | 2.43, 2.36 | 4.25 | 4.32 | 4.70, 4.32 |
| TTPαSe II | | | 7.81 | 1.97 | 6.36 | 2.42, 2.36 | 4.26 | 4.23 | 4.31 |

TABLE 2

¹³C-NMR chemical shifts (δ, ppm) of dNTPαSe analogs in D₂O

| Compound | C4 | C2 | C6 | C8 | C5 | C4′ | C1′ | C3′ | C5′ | C2′ |
|---|---|---|---|---|---|---|---|---|---|---|
| dATPαSeI | 159.97 | 156.26 | 153.34 | 141.04 | 119.33 | 86.33 | 84.40 | 71.73 | 66.17 | 39.70 |
| dATPαSeII | 156.43 | 153.53 | 152.21 | 141.12 | 119.33 | 86.46 | 84.23 | 71.88 | 66.57 | 39.95 |
| dGTPαSe I | 159.55 | 154.51 | 151.96 | 138.74 | 116.83 | 86.21 | 84.48 | 72.15 | 66.67 | 39.61 |
| dGTPαSe II | 159.70 | 154.59 | 152.05 | 138.60 | 116.86 | 86.26 | 84.42 | 72.01 | 66.62 | 39.60 |
| dCTPαSe I | 165.96 | 157.33 | 142.09 | | 96.68 | 85.93 | 85.17 | 70.66 | 65.36 | 39.36 |
| dCTPαSe II | 166.12 | 157.29 | 143.12 | | 97.30 | 86.75 | 86.07 | 71.55 | 66.50 | 40.23 |
| TTPαSe I | 167.50 | 152.64 | 138.31 | | 112.70 | 86.09 | 85.76 | 71.65 | 66.34 | 39.17 |
| TTPαSe II | 167.35 | 152.47 | 138.17 | | 112.49 | 85.95 | 85.53 | 71.35 | 66.58 | 39.23 |

TABLE 3

³¹P-NMR chemical shifts (δ, ppm) of dNTPαSe analogs in D₂O

| Compound | αP | γP | ßP |
|---|---|---|---|
| dATPαSeI | 33.78 | -7.35 | -23.38 |
| dATPαSeII | 33.47 | -7.57 | -23.40 |
| dGTPαSe I | 33.91 | -8.34 | -23.53 |
| dGTPαSe II | 33.58 | -7.96 | -23.53 |
| dCTPαSe I | 33.61 | -9.14 | -23.85 |
| dCTPαSe II | 33.56 | -9.91 | -24.28 |
| TTPαSe I | 33.31 | -7.48 | -23.67 |
| TTPαSe II | 32.64 | -7.14 | -23.58 |

### DNA Polymerization Reactions and Diastereomeric Activtiy

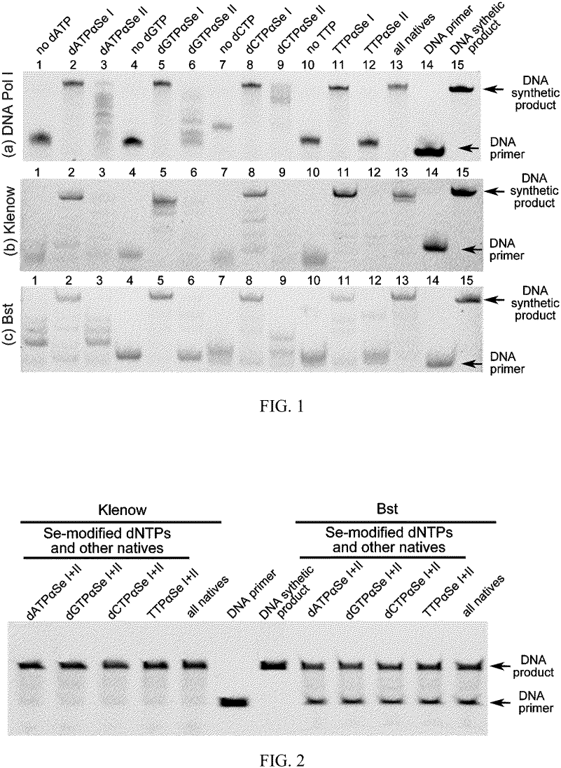

DNA polymerase extension and/or polymerization reactions with each Se-modified nucleotide diastereomer were performed with a 5′-FAM-labeled primer (DNA primer, 0.5 µM) and a template (DNA template, 0.5 µM), DNA polymerase [DNA polymerase I (DNA Pol I, 0.04 U/µL, NEB), Klenow Fragment (Klenow, 0.2 U/µL, NEB) or Bst Large Fragment (Bst, 0.3 U/µL, NEB)] and dNTPs (125 µM for DNA Pol I reactions, 15 µM for Klenow and Bst reactions). The reaction mixtures were incubated at 37° C. for 60 min, then equal volume of denaturing dye solution with 8 M urea was added into each tube. Complete termination was performed with an incubation in dry bath at 95° C. for 10 min. The products were analyzed by urea-denaturing polyacrylamide gel electrophoresis (Urea-PAGE) and imaging by FAM fluoresce, and compared to a DNA synthetic product. Results are shown in FIGS. 1-2.
DNA Primer: SEQ ID NO: 1; 15′-FAM-GTCGAGTCAAGAGCATCC-3′
DNA Template: SEQ ID NO: 2; 3′-
CAGCTCAGTTCTCGTAGGTTCAGCTAGGTCAGTCACATGAGTC-5′
Synthetic Product: SEQ ID NO: 3; 5′-FAM-
GTCGAGTCAAGAGCATCCAAGTCGATCCAGTCAGTGTACTCAG-3′

As shown in FIG. 1, the positive control with all native nucleotides gave the full-length DNA product, while the negative controls conduced in the absence of one native nucleotide did not generate any full-length product. We found that diastereomers I of the Se-modified nucleotides were efficiently recognized by DNA polymerases and incorporated into the extended strand. In contrast, diastereomers II of the Se-modified nucleotides were not recognized by the polymerases as substrates and substitution of the native nucleotides with diastereomer II of the corresponding modified nucleotide did not result in the extended DNA product for any of the three DNA polymerase enzymes. Extension and/or polymerization of the DNA fragment with Bst showed an almost identical product distribution as for mixtures lacking both the native and modified nucleotide all together (i.e., comparing between lanes 1 and 3, between lanes 4 and 6, between lanes 7 and 9, and between lanes 10 and 12 of FIG. 1).

Moreover, the presence of diastereomer II of the modified mucleotides did not inhibit the DNA polymerases. As shown in FIG. 2, substituting mixtures of modified nucleotide diastereomers had no noticeable effect on the DNA polymerization reaction in the presence of either Klenow or Bst enzymes. Unexpectedly, resolving each pair of the modified nucleotide diastereomers may not be necessary prior to their use as substrates in DNA polymerization reactions.

### Polymerization Rate and Specificity Using Selenium-Modified Nucleotides

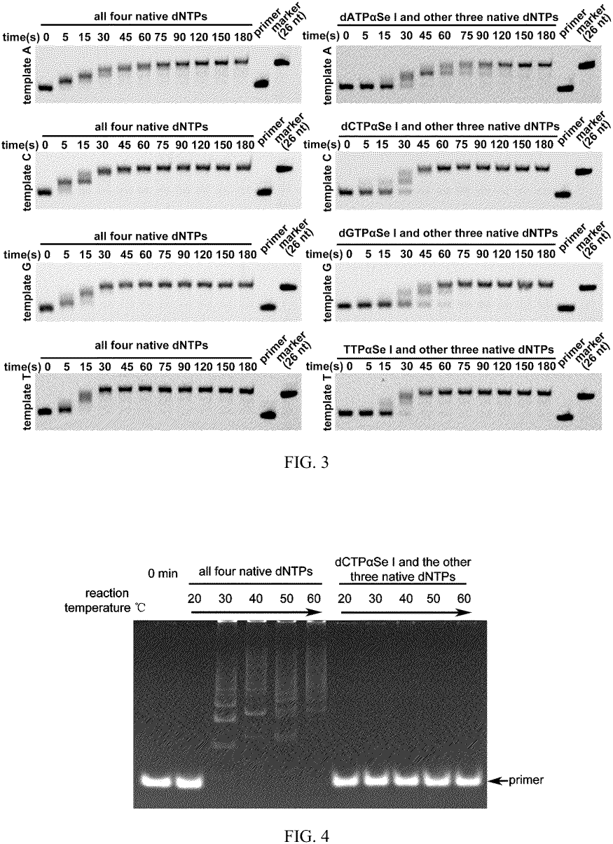

The reactions were performed with DNA primer (1 µM, final concentration), DNA template (0.7 µM), Bst (0.04 U/µL, NEB), a mixture of three native nucleotides and one Se-modified nucleotide (50 µM for each) and 1×Bst buffer (20 mM Tris-HCl, 10 mM (NH₄)₂SO₄, 10 mM KCl, 2 mM MgSO4, 0.1% Triton X-100 and 5 mM DTT) at 15 second intervals ranging from 0-180 sec, each at 55° C. Comparative examples were conducted for each template using only native nucleotides. The reactions were analyzed by denatured or native polyacrylamide gel electrophoresis and imaged by FAM-primer fluoresce (FIG. 3).
DNA Primer: SEQ ID NO: 1; 5′-FAM-GTCGAGTCAAGAGCATCC-3′
DNA Template (A): SEQ ID NO: 4; 3′-CAGCTCAGTTCTCGTAGGTCAGACAG-5′
DNA Template (C): SEQ ID NO: 5; 3′-CAGCTCAGTTCTCGTAGGGTCATCTA-5′
DNA Template (G): SEQ ID NO: 6; 3′-CAGCTCAGTTCTCGTAGGCATGTATG-5′
DNA Template (T): SEQ ID NO: 7; 3′-CAGCTCAGTTCTCGTAGGAATGTCTG-5′

As shown in FIG. 3, each of the polymerization reactions was slowed in the presence of a Se-modified nucleotide. Without being bound by theory, the decrease in the polymerization rate may be due to the replacement of an oxygen atom with a much larger selenium atom. In turn, the observed decrease in polymerization rate may improve the fidelity of the DNA polymerization, without significantly reducing yields for processes such as PCR that employ exponential amplification curves.

### Inhibition of Non-Specific DNA Primer Extension

In addition to increased fidelity, the reduced polymerization rate for Se-modified nucleotides was shown to reduce non-specific extension products in the polymerization reactions. The reactions using Se-modified nucleotides to inhibit non-specific DNA primer extension were performed with DNA primer (2 µM, final concentration), DNA template (2 µM), Bst (0.5 U/µL, NEB) and native and/or Se-modified nucleotides (200 µM each). Extension and polymerization in the absence of DNA template were conducted at temperatures ranging from 20-60° C. (20, 30, 40, 50 and 60° C., FIG. 4). Based on those preliminary results, and the optimal temperature of Bst (55° C.), the DNA primer extensions in the presence of DNA template were conducted at 55° C. for 60 minutes. The reactions were repeated for 90 minutes. Each of the reactions were analyzed by urea-denaturing or native polyacrylamide gel electrophoresis and imaged by FAM-primer fluoresce visualizing primer extension on denaturing gel, or gel-red staining visualizing all extended products on native gel.

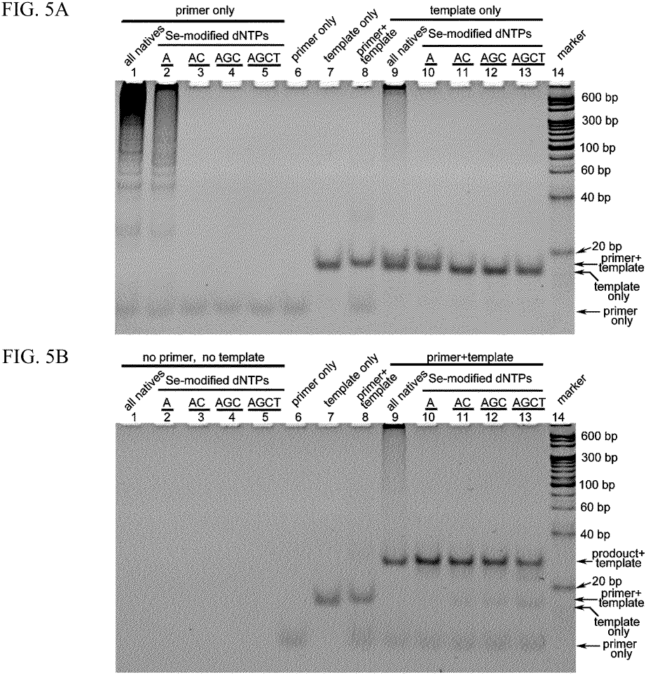

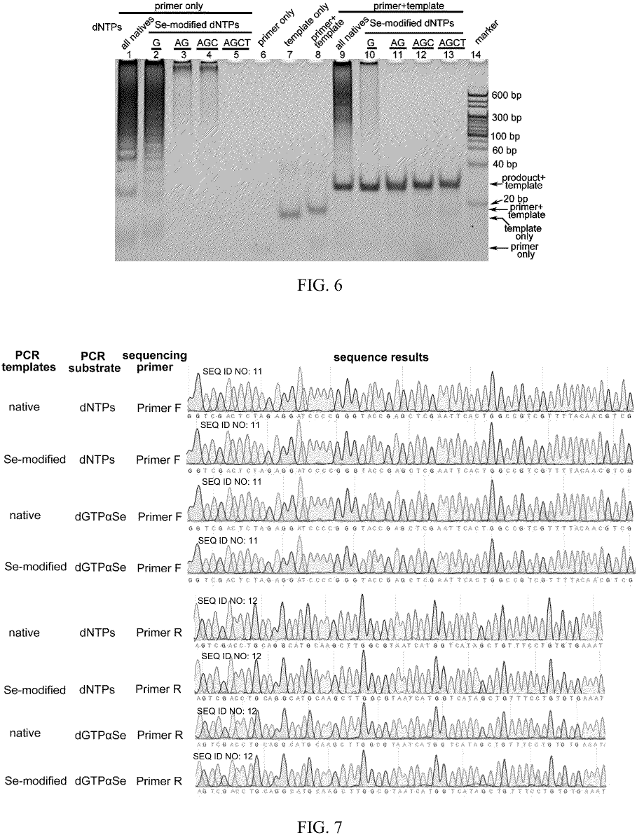

As shown in FIGS. 5-6, native nucleotides caused nonspecific DNA polymerization in both the presence and absence of a DNA template, and generated multiple by-products (especially longer by-products, FIG. 5A). Unexpectedly, we found that substitution of Se-modified nucleotides (diastereomer I) for one or multiple native nucleotides offered much cleaner polymerization (both in the presence and absence of DNA template) and similar yield. Since the primer was not labeled, these PAGE gels were stained by GelRed visualizing the generated DNAs in the reaction samples. The lack of signals (lanes 3-6) in the “primer only” experiments (FIG. 5A) was due to the complete suppression of non-specific extension and/or polymerization by the Se-modified nucleotides, especially in the presence of multiple Se-modified nucleotides. The de novo DNA synthesis by DNA polymerase was not observed in the absence of DNA (FIG. 5B). Moreover, we observed that substitution with a single Se-modified nucleotide effectively suppressed by-product formation.

Surprisingly, substitution with multiple Se-modified nucleotides completely prevented non-specificity, without significant loss synthetic efficiency. DNA polymerase was still functional, even all four native nucleotides were replaced with Se-modified nucleotides, and the yield remained the same. Obviously, the Se-modified nucleotides provide much higher specificity in DNA primer extension and polymerization than their native counterparts.

### PCR and Sequencing of Selenium-Modified DNA

To monitor the PCR amplification with Se-modified nucleotide substrates and/or Se-DNA templates, we first prepared Se-modified DNA by using Se-modified nucleotides, a native template DNA (SEQ ID NO: 8; 5′-acgacgttgtaaaacgacggccagtgaattcgagctcggtacccggggatcctctagagtcgacctgcaggcatgcaagcttggcgtaatc atgg-tcat-3′), and a Primer T (SEQ ID NO: 9; 5′-aatttcacacaggaaacagctatgaccatgattacgcc-3′). The polymerized Se-DNA was purified on urea-PAGE gel (12.5%) and the purified Se-DNA was used as the template for 30 cycles of PCR amplification with Se-modified and/or native nucleotide substrates (0.2 mM for each), 0.6 µM primers, 0.15 U/µl Taq DNA polymerase and 2 mM Mg²⁺. Then we performed sequencing with both forward and reversed primers. The results shown in FIG. 6 demonstrate that the Se-modified nucleotides and Se-modified DNA can be directly used as regular substrates and DNA templates for sequencing, respectively.

### Oxidative Deselenization of Selenium-Modified DNA

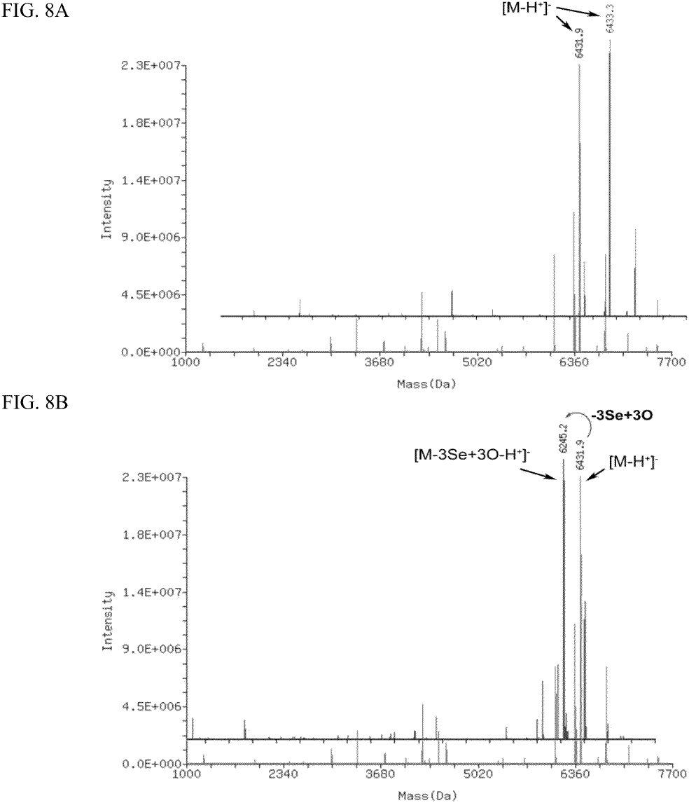

Oxidation of Se-DNA with hydrogen peroxide yielded the corresponding native DNA. Single-stranded native DNA and Se-modified DNAs were prepared via an exponential amplification reaction (EXPAR). The two resulting DNA sequences were purified by PAGE and desalted by C18 cartridges (Sep-Pac Vac, Waters Co.). Then the Se-DNA was treated, with 3% fresh H₂O₂ for deselenization at room temperature for 24 hours, or alternatively, at 50° C. for 2 hours. The resulted samples were analyzed by ESI-MS. The sequence of the single-stranded Se-DNA is SEQ ID NO: 10; 5′-d(pApGpTpApCpTpApGpApTpGpTpGpApGpApCpApTpC), containing dC-phosphoroselenoate. Se-DNA molecular formula: C₁₉₇H₂₄₇N₇₉O₁₁₆P₂₀Se₃, [M-H⁺]⁻:6432.8 (calc.); fully-deselenized Se-DNAs (corresponding native DNA) molecular formula: C₁₉₇H₂₄₇N₇₉O₁₁₉P₂₀, [M-3Se+3O-H⁺]⁻: 6243.1 (calc.). A: Overlapped MS profiles of Se-DNAs treated with and without H₂O₂ at room temperature for 24 h (profiles in red and gray, respectively) and observed mass are 6431.9 (6432.8, calc.) and 6433.3 (6432.8, calc.), respectively; B: Overlapped MS profiles of Se-DNAs treated with and without H₂O₂ at 50° C. for 2 h (profiles in red and gray, respectively) and observed mass are 6431.9 (6432.8, calc.) and 6243.1 (6245.2, calc.), respectively. As apparent from FIG. 8, the selenium modifications were not removed at room temperature; however the selenium modifications were completely removed to yield the native DNA upon mild heating. Accordingly, the selenium derivatives disclosed herein are stable at room temperature, and can be conveniently converted to the corresponding native DNA under mild conditions.

### Preparation And Evaluation Of Alpha-Phosphate Thio Modified Nucleotides (dNTPαS) In Enzymatic Extension Reactions

As further non-limiting examples, dNTPαS were prepared as disclosed in Caton-Williams, J.; Fiaz, B.; Hoxhaj, R.; Smith, M.; Huang, Z., Convenient synthesis of nucleoside 5′-(α-P-thio)triphosphates and phosphorothioate nucleic acids (DNA and RNA). J Science China Chemistry 2012, 55 (1), 80-89, which is hereby incorporated herein in its entirety by reference. Again, diastereomers of each dNTPαS were evaluated separately with DNA polymerase, Klenow fragment, and Bst polymerase, and it was found that the dNTPαS I diastereomers were efficiently recognized by the extension and/or polymerization enzymes, while the dNTPαS II diastereomers were not recognized by the enzymes.

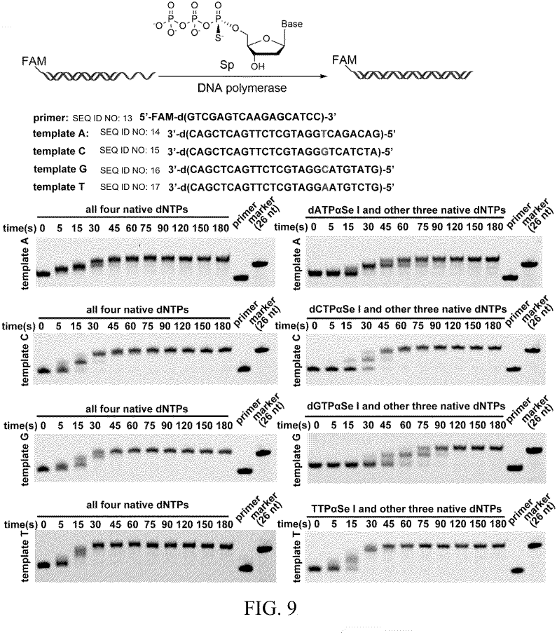

Surprisingly, as for dNTPαSe nucleotides discussed above, the dNTPαS nucleotides exhibited a delayed incorporation into the primer sequence by Bst polymerase compared with the naturally occurring native nucleotides, as shown in FIG. 9.

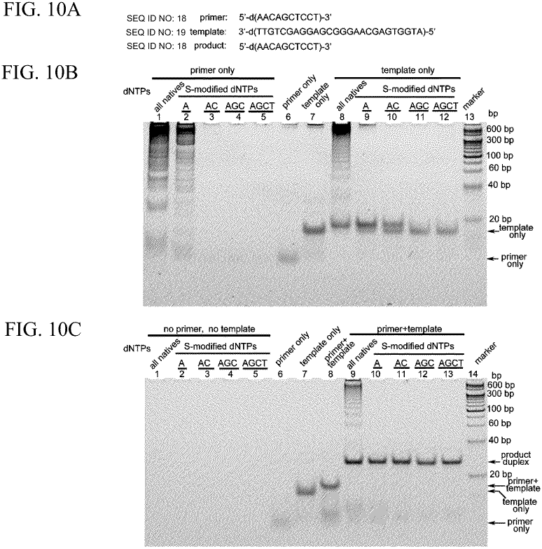

To study the suppression of nonspecific amplification in the present and absence of template or/and primer, we designed primer extension with/without template or/and primer, since primers can use each other as nonspecific templates, thereby generating nonspecifically extended by-products, such as primer dimers and background-synthesized DNA by-products. In the absence of template or primer, it was observed that nonspecific DNA extensions occurred, indicated by formation of multiple by-products (FIG. 10B). And in the presence of DNA template, native dNTPs caused nonspecific DNA polymerization and generated multiple by-products too (FIG. 10C). Besides, the results of no nucleic acid reactions indicated that the nonspecific products observed in FIGS. 10B and 10C did not result from de novo synthesis of polymerase without any nucleic acid. On the contrary, and unexpectedly, dNTPαS I substitution (one or multiple dNTPs) offered much cleaner polymerization (both in the presence and absence of DNA template) and similar yield. We observed that single dNTPαS I substitution could effectively suppress by-product formation. Furthermore, it appears that multiple S-dNTPs can completely prevent the nonspecificity, with similar synthesis efficiency. In addition, we also found nonspecific products resulting from smaller sequence (primer) were much more and more difficult to inhibit completely (FIG. 10B). Interestingly, DNA polymerase was still functional, even all four native dNTPs were replaced with four dNTPαS I, while the yield remained the same. Obviously, the S-modified dNTPs disclosed herein provided much higher specificity in DNA primer extension and polymerization than the native dNTPs.

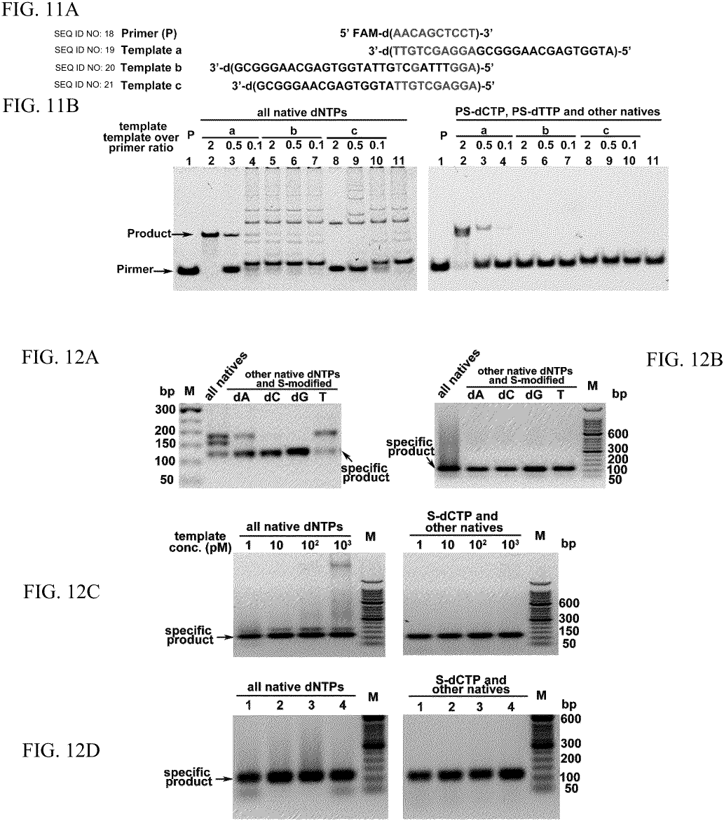

To verify the nonspecific amplification inhibition effect of S-dNTP at various paring conditions, we designed primer extension (FIG. 11) with extensible template (Template a), poor-paired template (Template b) and inextensible but pairable template (Template c) at various concentrations. The result indicated that the nonspecific amplification was inhibited when the extensible template (Template a) and Primer can completely match and produce specific extension products. However, when “stabilizer” (template) was much less than “reactive” primer, nonspecific products were generated (FIG. 11B). Further, as Template b could not pair Primer well, nonspecific production was not inhibited within the range of 0.1-2 equivalence. Furthermore, in the reactions with Template c, which could completely pair but cannot generate extended product, increased Template c to 2 equivalence also significantly inhibited the generation of nonspecific amplification. In addition, reaction without template produced much nonspecific product. But, encouragingly, in the reactions of PS-dNTPs as substrate, nonspecific amplification can be completely inhibited at all conditions above-mentioned (FIG. 11).

Compared to Bst DNA polymerase, Taq DNA polymerase is used much more widely, such as in polymerase chain reaction (PCR). In PCR reactions amplifying plasmid (FIG. 12A) and total cDNA (FIG. 12B), we surprisingly found that dNTPαS I can suppress nonspecific product generation obviously, without having a negative effect on PCR yield. Further, suppression effect of each dNTPαSe I on nonspecific product was varied in reactions for amplifying simple plasmid (FIG. 12A) and complex template (total cDNAs, which were reverse-transcribed from cell total RNA, so generating nonspecific product in PCR using cDNAs as template is straightforward without iterating and optimizing reactions conditions (FIG. 12B). All dNTPαS I exhibited an inhibiting effect, especially dCTPαS I and dGTPαSe I which suppressed nonspecific product nearly completely and generated a more specific product. Furthermore, the suppression of PCR nonspecific product was widely effective at various template (pEGFP in FIGS. 12C, 12E; total cDNA in FIGS. 12D, 12F) concentrations.

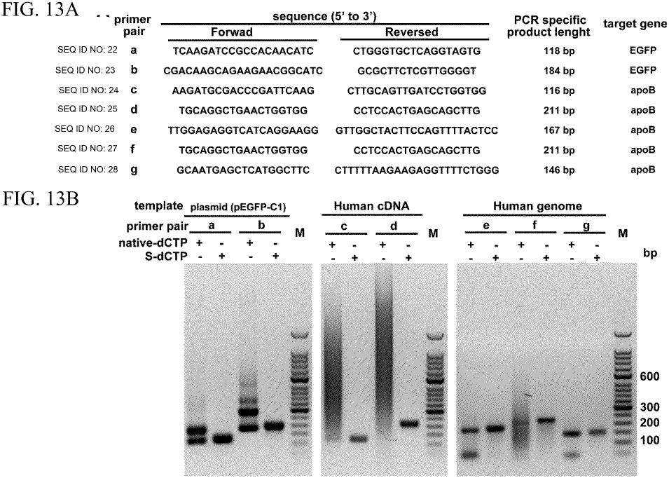

To test universality of suppression effect of dNTPαS I on nonspecific product, we checked 5 new pairs of primers (primer pair a, b, c, d, e) and 3 types of templates (plasmid, total human cDNA and human genome) with native dNTPs or S-modified dNTPs (FIG. 13). The results indicated that all reactions using 4 native dNTPs generated obvious nonspecific products which can be nearly completely suppressed when S-dCTP replaced native dCTP at all conditions we tested. Further, S-dNTP can inhibit nonspecific amplification and increase specific product when amplifying simple plasmid template (FIG. 13B). Furthermore, when amplifying complex template (cDNA or genome), reactions using S-dNTP could exact specific product which can not be synthesized using all native dNTPs (FIG. 13B).

### Preparation And Evaluation Of Base Modified dTTP Nucleotides

Modified nucleotides having Se (or S) modifications at the 2-position of the thymine base were prepared according the procedures described below.

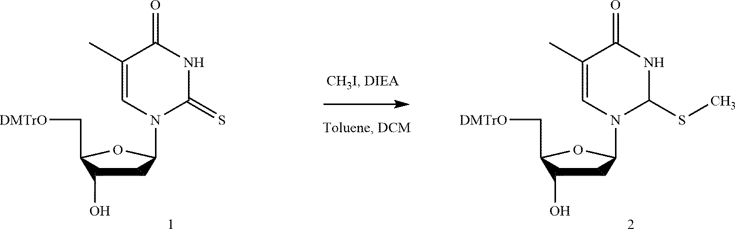

One grams of dry compound 1 was dissolved in dry dichloromethane (DCM, 10 ml), followed by addition of toluene (5 ml). N,N-diisopropylethylamine (DIEA, 0.69 g) and iodomethane (0.76 g)was then added to the reaction mixture at room temperature. The reaction was monitored by thin layer chromatography (TLC) plate (10% methanol in dichloromethane, R∱= 0.4) and completed in 1h. Methanol (5 ml) was poured into the mixture and stirred for 5 minutes to quench the reaction. The organic phase was evaporated under reduced pressure, DCM (50 ml) was then added. The organic layer was washed once with ddH₂O (50 ml) and then three times with Saturated sodium bicarbonate aqueous solution (50 ml). The organic phase was dried over anhydrous magnesium sulfate and evaporated under reduced pressure. The residue was purified by flash column chromatography (5% methanol in dichloromethane) and pure compound 2 was obtained in 89% yield.

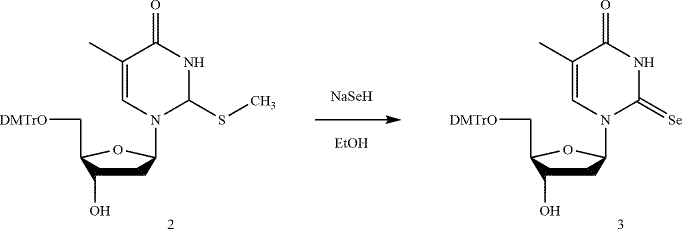

A solution of NaSeH was generated by addition of absolute ethanol (5 ml) to selenium (0.41 g) and sodium borohydride (NaBH₄, 0.25 g) at 0° C. The reaction was completed in 0.5 h and a clear solution was formed. The ethanolic solution was added to compound 2 (0.5 g) and the mixture was stirred overnight under argon. The reaction mixture was then concentrated under reduced pressure and ethyl acetate (5 ml) was added to the residue. The organic layer was washed with water three times (3 × 30 ml), and then dried over anhydrous magnesium sulfate. Purification was performed by flash column chromatography (5% methanol in dichloromethane) and the light yellow compound 3 was obtained in 83% yield.

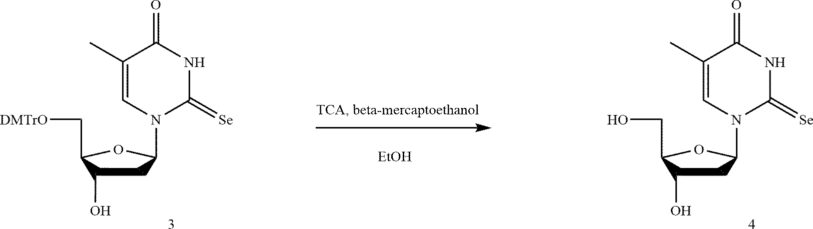

Compound 3 (0.22 g) was added to a 10 ml round bottom flask, then 2 ml DCM was added to dissolve it, followed by addition of mercaptoethanol (0.2 g). Trichloroacetic acid (TCA) solution (10% TCA in DCM) was cautiously added dropwise until the mixture turned orange. Then the mixture was stirred for 10 minutes, and the reaction was monitored by TLC plate (10% methanol in dichloromethane, R∱ = 0.3). Wash the solid with DCM under vacuum filtration and the white compound 4 was obtained in 95% yield.

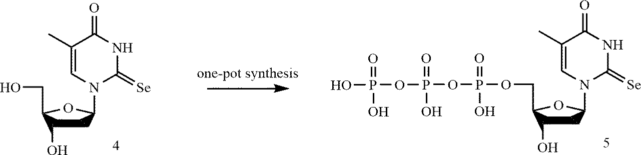

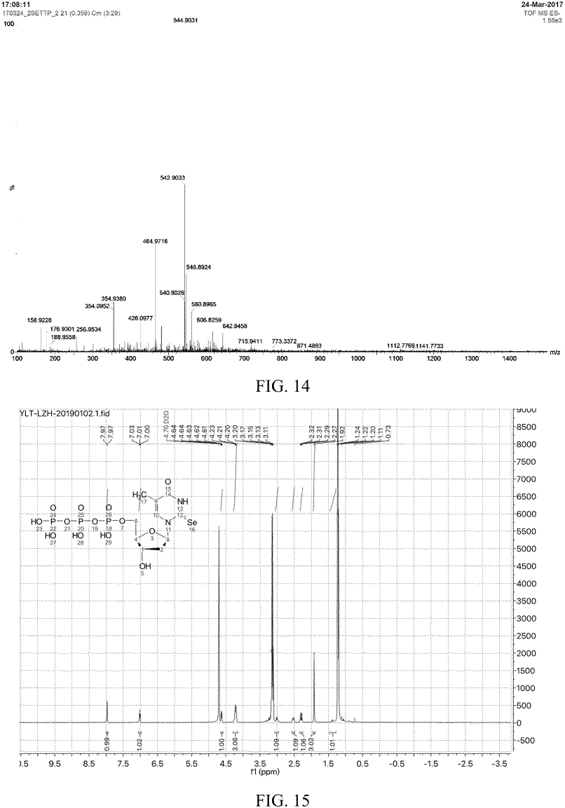

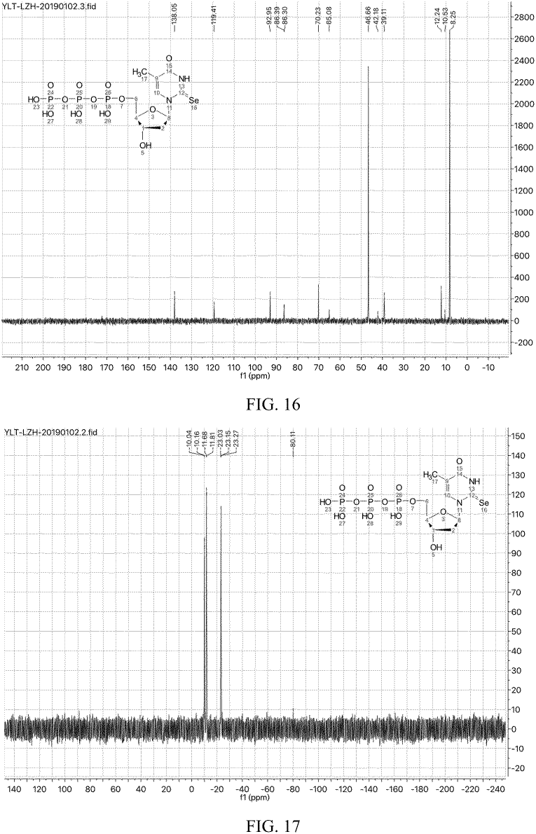

Tributyl ammonium pyrophosphate (170.4 mg) was added into a 25 mL round bottom flask and dried overnight. 2-chloro-4H-1,3,2-benzodioxin-4-one (33.3 mg) was put into a 5 ml round bottom flask and dried with a vacuum pump for 15 minutes. After argon replacement, 0.3 ml anhydrous DMF and 0.6 ml anhydrous tri-n-butylamine was added by a syringe to dissolve tributyl ammonium pyrophosphate under argon (Reagent 1). 2-chloro-4H-1,3,2-benzodioxin-4-one was dissolved in 0.6 ml anhydrous DMF .Then it was added into Reagent 1 with another syringe and stirred for 30 minutes under argon (Reagent 2). Compound 4 (52.3 mg) was dissolved in 0.3 ml anhydrous DMF and then moved into Reagent 2 by a new syringe. The reaction was stirring for 1h under argon (Reagent 3). Iodine (30 mg) was dissolved in 5 ml anhydrous DMF and then added to Reagent 3 dropwise until the mixture showed a bright yellow color that did not fade. Then the reaction was stirring for 0.5 h under argon. Triethylamine (54 mg) and degassed water (5 ml) were added to the mixture, and argon was used to protect the reaction for 1.5 h. DTT (50 ul) was added into the mixture by syringe to quench the reaction. After three times precipitation with ethanol, Purification was performed by HPLC and compound 5 was obtained in 12% yield. Compound 5, 2-Se-dTTP, was characterized by HPLC, HRMS, and ¹H, ¹³C, and ³¹P NMR, according to FIGS. 14-17.

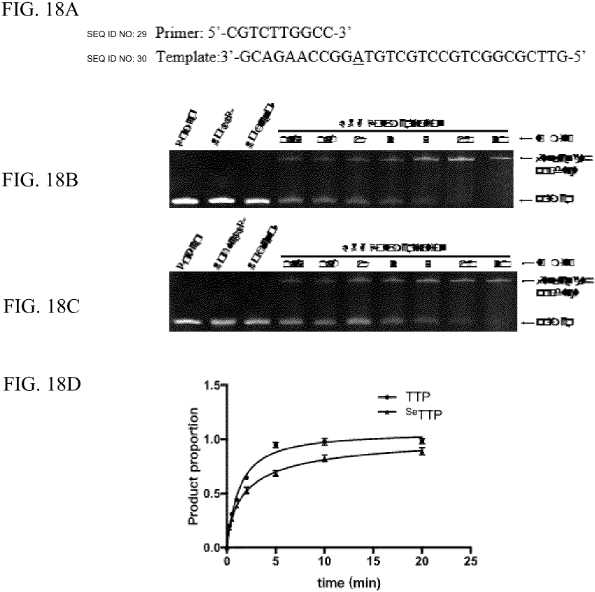

As shown in FIG. 18, no full-length DNA was observed in the control experiment where TTP (or 2-Se-TTP) was absent, as well as other two control experiments. The 30-nt DNA products made from the polymerization using TTP and 2-Se-TTP refer to full-length product in FIGS. 2B and 2C, respectively. We observed that 2-Se-TTP was relatively well recognized by the Klenow fragment of DNA polymerase I and that the recognition was almost as good as that for TTP (FIG. 18). Results of thermal dynamic calculation (FIG. 18) are consistent with the denatured PAGE. The initial reaction rate ratio (TTP vs 2-Se-TTP) was around 1.24.

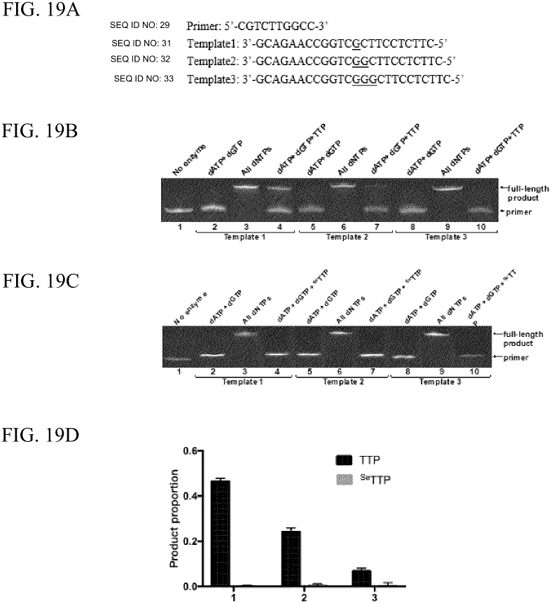

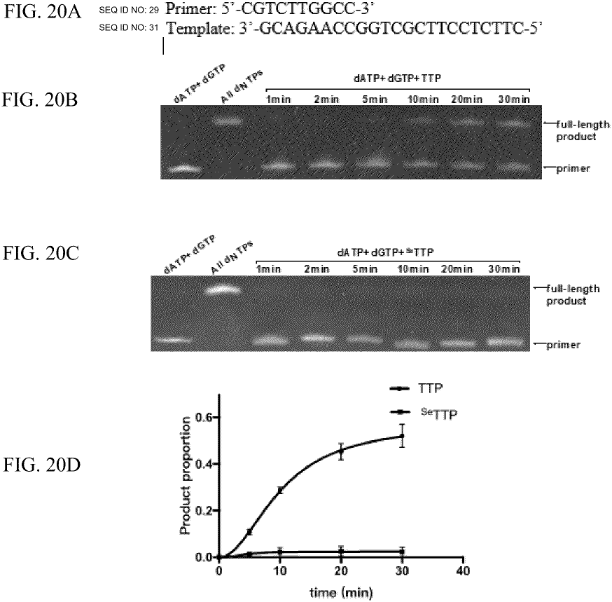

Experiments to examine the misincorporation of 2-Se-TTP were conducted as follows. Broadly, enzymatic extension reactions were performed using each naturally occurring dNTP except dCTP, knowing that misincorporations of TTP in place of dCTP are commonly the cause of decreased fidelity in DNA replications. As shown in FIGS. 18-21, where dCTP was not present, native TTP was incorporated into the DNA primer sequence in a significant amount. In contrast, when 2-Se-TTP was used, there was little to no full-length product produced, even after 30 minutes (as shown by FIG. 20).

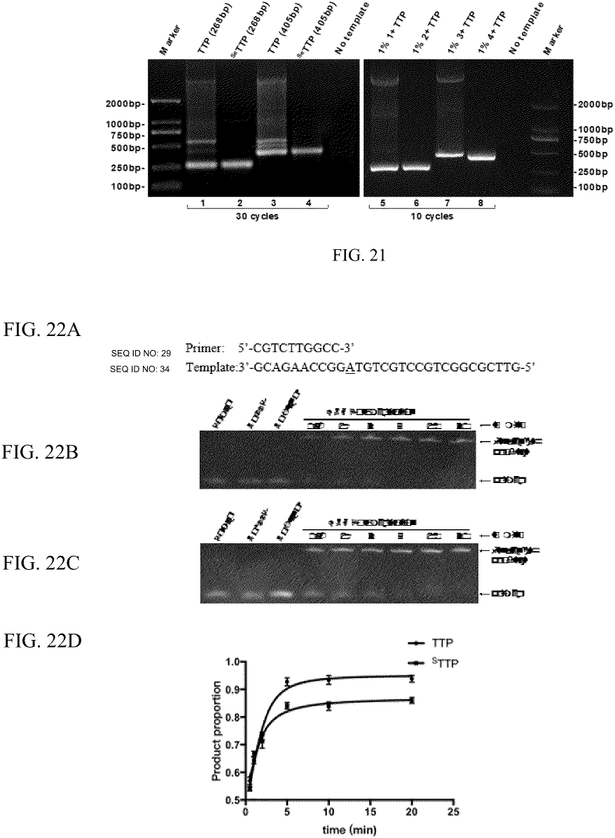

To further confirm the application value of 2-Se-TTP in PCR, two different target fragments were designed, shown in FIG. 21 (268bp, 405bp). The results of agarose gel show that nonspecific amplification bands were observed in both of the control experiments (FIG. 21), while clean and specific bands were obtained both in the experimental groups where 2-Se-TTP was additionally added. No bands were found in two blank groups. Thus, incorporation of 2-Se-TTP can effectively reduce the non-specific bands in PCR. Sequencing results showed that there was no significant difference in 1, 2, 5 and 6, as well as 3, 4, 7 and 8.

Now our biological experiments reveal that polymerase is able to recognize and utilize 2-Se-TTP, and with the incorporation of 2-Se-TTP in DNAs, the specificity of the base pair recognition is largely increased, which lead to more specific DNA bands in polymerase reactions. Consistently, our experimental results also indicate that the incorporation of 2-Se-TTP can indeed reduce the nonspecific amplification of bands in PCR, which providing a unique strategy for PCR optimization. Moreover, this 2-Se-TTP provides a brand-new approach for further investigating base-pair recognition and DNA polymerase replication, while this 2-Se-UTP provides a brand-new approach for further investigating base-pair recognition and RNA polymerization, opening new research opportunities for RNA polymerase transcription, reverse transcription, and mRNA translation.

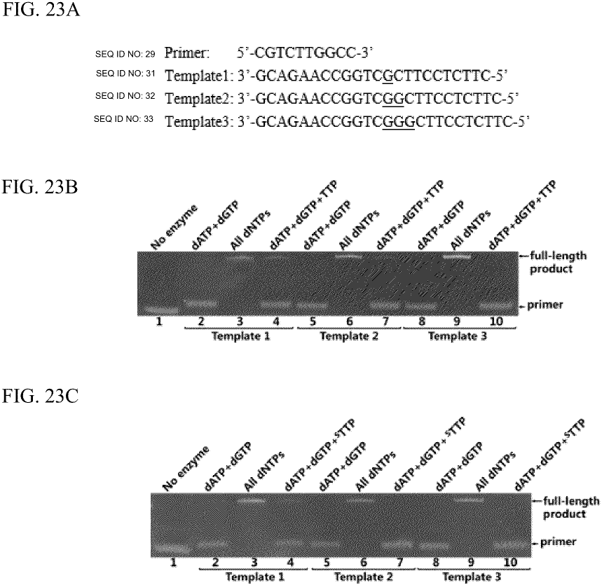

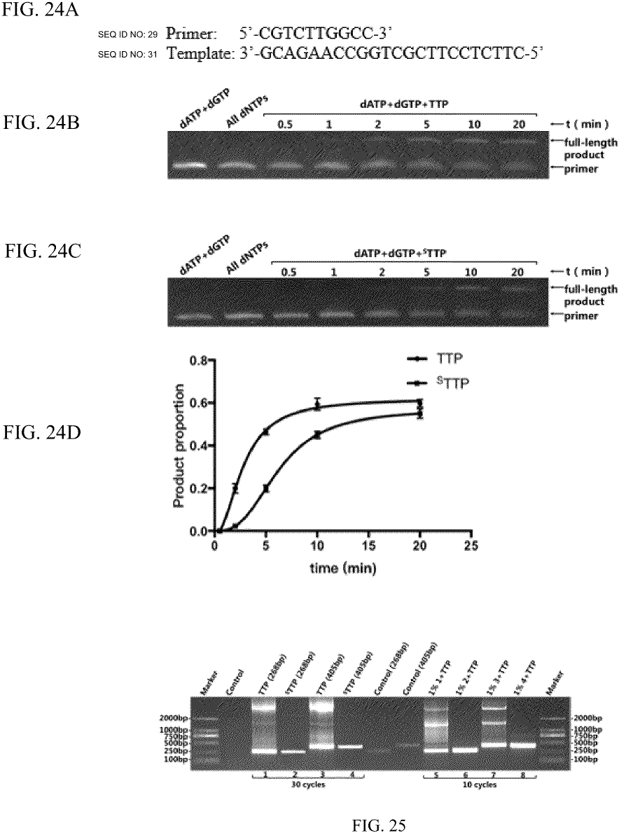

Exploring further the potential of base modifications at the 2-position of thymine, analogous experiments were conducted using 2-S-TTP as a modified nucleotide with results shown in FIGS. 22-25. As is shown, sulfur modifications had a similar advantageous effect to the specificity of DNA replication compared to native nucleotides. Likewise, this 2-S-UTP provides a brand-new approach for further investigating base-pair recognition and RNA polymerization.

The invention is described above with reference to numerous aspects and specific examples. Many variations will suggest themselves to those skilled in the art in light of the above detailed description. All such obvious variations are within the full intended scope of the appended aspects. Other aspects of the invention can include, but are not limited to, the following (aspects are described as “comprising” but, alternatively, can “consist essentially of” or “consist of”):
Aspect 1. An enzymatic process for forming a nucleic acid product mixture, the process comprising:
   annealing a primer or promoter sequence to a template sequence; and
   extending the primer sequence or synthesizing a nucleic acid in the presence of an extension and/or polymerization enzyme and a nucleotide mixture comprising at least one modified nucleotide to form a modified nucleic acid;
   wherein an amount of nonspecific byproducts in the product mixture due to mispriming of the primer or promoter sequence during the annealing step, or misincorporation of the modified nucleotide during the extending or synthesizing step, is less than that of an otherwise identical process using a native nucleotide.
Aspect 2. The process of aspect 1, further comprising isolating the modified nucleic acid.
Aspect 3. The process of aspect 1, further comprising oxidizing or hydrolyzing the modified nucleic acid to produce an analogous native nucleic acid.
Aspect 4. The process of aspect 3, wherein oxidizing the modified nucleic acid comprises heating the modified nucleic acid in the presence of an oxidant.
Aspect 5. The process of aspect 4, wherein the oxidant is dilute hydrogen peroxide.
Aspect 6. The process of aspect 1, wherein the process is a cDNA synthesis, a PCR amplification, an isothermo amplification, or a sequencing process.
Aspect 7. The process of aspect 3, wherein the analogous native nucleic acid is a DNA.
Aspect 8. The process of aspect 1, wherein the native nucleotide comprises a naturally-occurring base.
Aspect 9. The process of aspect 1, wherein the native nucleotide comprises a base modified with a fluorescent moiety, a gamma-phosphate modified with a fluorescent moiety, or both.
Aspect 10. The process of aspect 9, wherein the native nucleoside is any native sequencing nucleotide disclosed herein, e.g., 3′-O-N₃-dATP, 3′-O-N₃-dCTP, 3′-O-N₃-dGTP, 3′-O-N₃-dTTP, ddCTP-N₃-Bodipy-FL-510, ddUTP-N₃-R6G, ddATP-N₃-ROX, ddGTP-N₃-Cy5, or combinations thereof.
Aspect 11. The process of aspect 1, wherein the modified nucleotide comprises an Se-modified nucleotide, an S-modified nucleotide, or both.
Aspect 12. The process of aspect 1, wherein the nucleotide mixture comprises more than one modified nucleotide.
Aspect 13. The process of aspect 12, wherein the modified nucleotide is a mixture of diastereomers.
Aspect 14. The process of aspect 1, wherein the modified nucleotide is a modified deoxyribonucleotide triphosphate (dNTP) or a ribonucleotide triphosphate (NTP).
Aspect 15. The process of aspect 1, wherein the modified nucleotide is dATPαS, dGTPαS, dCTPαS, TTPαS (or dTTPαS), dUTPαS, 2-S-TTP (or 2-S-dTTP), 2-S-dUTP, 2-S-TTPαS (or 2-S-dTTPαS), 2-S-dUTPαS, ATPαS, GTPαS, CTPαS, UTPαS, rTTPαS, 2-S-UTP, 2-S-rTTP, 2-S-UTPαS and 2-S-rTTPαS. Aspect 16. The process of aspect 1, wherein the modified nucleotide is dATPαSe, dGTPαSe,dCTPαSe, TTPαSe (or dTTPαSe), dUTPαSe, 2-Se-TTP (or 2-Se-dTTP), 2-Se-dUTP, 2-Se-TTPαSe (or 2-Se-dTTPαSe), 2-Se-dUTPαSe, ATPαSe, GTPαSe, CTPαSe, UTPαSe, rTTPαSe, 2-Se-UTP, 2-Se-rTTP, 2-Se-UTPαSe and 2-Se-rTTPαSe.
Aspect 17. The process of aspect 1, wherein the modified nucleotide is 2-thio-dCTP, 2-thio-CTP.
Aspect 18. The process of aspect 1, wherein the modified nucleotide is 2-seleno-dCTP, 2-seleno -CTP.
Aspect 19. The process of aspect 1, wherein the modified nucleotide is any modified sequencing nucleotide disclosed herein, e.g., 3′-O-N₃-dATPαSe, 3′-O-N₃-dGTPαSe, 3′-O-N₃-dCTPαSe, 3′-O-N₃-dTTPαSe, 3′-O-N₃-dUTPαSe, ddCTPαSe-N₃-Bodipy-FL-510, ddUTPαSe-N₃-R6G, ddATPαSe-N₃-ROX, ddGTPαSe-N₃-Cy5, 3′-O-N₃-dATPαS, 3′-O-N₃-dCTPαS, 3′-ON₃-dGTPαS, 3′-O-N₃-dTTPαS, 3′-O-N₃-dUTPαS, ddCTPαSe-N₃-Bodipy-FL-510, ddUTPαSe-N₃-R6G, ddATPαSe-N₃-ROX, ddGTPαSe-N₃-Cy5, or combinations thereof.
Aspect 20. The process of aspect 1, wherein the nucleotide mixture comprises at least one native nucleotide (e.g., from 1 to 4 native nucleotides).
Aspect 21. The process of aspect 20, wherein the native nucleotide is selected from the group comprising dATP, dGTP, dCTP, TTP, dUTP, ATP, GTP, CTP, UTP, rTTP, and combinations thereof.
Aspect 22. The process of aspect 1, wherein the nucleotide mixture comprises more than one native nucleotide.
Aspect 23. The process of aspect 1, wherein the nucleotide mixture comprises a modified nucleotide and the analogous native nucleotide.
Aspect 24. The process of aspect 23, wherein a molar ratio of the modified nucleotide to the analogous native nucleotide is in any range disclosed herein (e.g., from 1:100 to 10:1, from 1:10 to 10:1, from 1:5 to 5:1, from 1:2 to 10:1), or analogous native nucleotide may not even be included in the reagent mixture.
Aspect 25. The process of aspect 1, wherein a concentration of each modified nucleotide, each native nucleotide, and/or each combination of modified nucleotide and the analogous native nucleotide, in the nucleotide mixture during the extending step, independently is in any range disclosed herein (e.g., from about 1 fM to about 100 mM, from about 50 µM to about 300 µM, etc.).
Aspect 26. The process of aspect 1, wherein the extension and/or polymerization enzyme comprises any enzyme or fragment thereof disclosed herein (e.g., a DNA polymerase, an RNA polymerase, a reverse transcriptase).
Aspect 27. The process of aspect 26, wherein an extension and/or polymerization rate of the extension and/or polymerization enzyme for the modified nucleotide is less than that for an analogous native nucleotide.
Aspect 28. The process of aspect 26, wherein an extension and/or polymerization rate of the extension and/or polymerization enzyme for the modified nucleotide is in any range disclosed herein (e.g., from about 1 base pairs/second to about 10,000 base pairs per second, from about 1000 to about 8000 base pairs per second, from about 2000 to about 6000 base pairs per second, from about 3000 to about 5000 base pairs per second), or is any amount or percentage less than that relative to an analogous extension and/or polymerization rate of the extension and/or polymerization enzyme for native nucleotides (e.g., about 90 %, about 80%, about 60%, about 50%, about 30%, about 20%, about 10% or about 1% less than the analogous extension and/or polymerization rate, or at least about 1 base pairs per second, at least about 500 base pairs per second, or at least about 1000 base pairs per second less than the analogous extension and/or polymerization rate.
Aspect 29. The process of aspect 1, wherein an error rate of the extension and/or polymerization enzyme for the modified nucleotide is less than that for the analogous native nucleotide.
Aspect 30. The process of aspect 1, wherein an error rate of the extension and/or polymerization enzyme for the modified nucleotide is at least 10% less than that for the analogous native nucleotide.
Aspect 31. The process of aspect 1, wherein an error rate of the extension and/or polymerization enzyme is less than about 1 per 10⁵ base pairs.
Aspect 32. The process of aspect 31, wherein the error rate of the extension and/or polymerization enzyme is less than about 1 per 10⁶ base pairs.
Aspect 33. The process of aspect 1, wherein the annealing and extending steps are repeated for any number of cycles disclosed herein (e.g., from about 20 to about 40 cycles).
Aspect 34. The process of aspect 1, wherein an amount of error-free modified nucleic acid in the product mixture is higher than that of an otherwise identical process using an analogous native nucleotide.
Aspect 35. A reagent mixture for conducting nucleic acid extension and/or polymerization reactions, the mixture comprising:
   a DNA primer sequence;
   a DNA template sequence;
   a DNA polymerase enzyme; and
   a nucleotide mixture comprising:
      at least one native nucleotide selected from the group consisting of dATP, dGTP, dCTP, TTP and dUTP; and
      at least one Se-modified or S-modified nucleotide.
Aspect 36. The mixture of aspect 35, further comprising a reaction buffer.
Aspect 37. The mixture of aspect 36, wherein the reaction buffer comprises a salt selected from the group consisting of a Mg salt, a Co salt, a Mn salt, a Cd salt, a Zn salt, or any combination thereof.
Aspect 38. The mixture of aspect 36, wherein the reaction buffer comprises Tris-HCl, (NH₄)₂SO₄, 10 mM KCl, 2 mM MgSO₄, 0.1% Triton® X-100, or any combination thereof.
Aspect 39. The mixture of aspect 36, wherein a pH of the reaction buffer is in a range from about 5 to about 10.
Aspect 40. The mixture of aspect 35, wherein the template sequence comprises Se-modified nucleotides.
Aspect 41. The mixture of aspect 35, wherein the length of the template sequence is any length disclosed herein (e.g., at least about 3 base pairs, at least about 100 base pairs, at least about 1,000 base pairs, in a range from about 3 to about 10,000 base pairs, etc.).
Aspect 42. The mixture of aspect 35, wherein a concentration of the template sequence is in any range disclosed herein (e.g., from about 1 yoctoM to about 1 mM).
Aspect 43. The mixture of aspect 35, wherein the primer sequence comprises Se-modified nucleotides.
Aspect 44. The mixture of aspect 35, wherein the length of the primer sequence is any length disclosed herein, e.g., in a range from about 3 to about 100 nt.
Aspect 45. The mixture of aspect 35, wherein a concentration of the primer sequence is in any range disclosed herein (e.g., from about 1 yoctoM to about 100 mM).
Aspect 46. The mixture of aspect 35, wherein a ratio of the concentration of primer sequence to that of the template sequence is in any range disclosed herein (e.g., from about 1:2 to about 1,000:1).
Aspect 47. The mixture of aspect 35, wherein the extension and/or polymerization enzyme is any DNA polymerase or an enzymatically active fragment thereof disclosed herein (e.g., Bst polymerase, Klenow fragment, etc.).
Aspect 48. The mixture of aspect 35, wherein a concentration of the extension and/or polymerization enzyme is in any range disclosed herein, e.g., from about 0.0001 U/µL to about 1,000 U/µL.
Aspect 49. The mixture of aspect 35, wherein the Se-modified nucleotide is any modified sequencing nucleotide disclosed herein, e.g., 3′-O-N₃-dATPαSe, 3′-O-N₃-dGTPαSe, 3′-O-N₃-dCTPαSe, 3′-O-N₃-dTTPαSe, 3′-O-N₃-dUTPαSe, ddCTPαSe-N₃-Bodipy-FL-510, ddUTPαSe-N₃-R6G, ddATPαSe-N₃-ROX, ddGTPαSe-N₃-Cy5, 3′-O-N₃-dATPαS, 3′-O-N₃-dCTPαS, 3′-ON₃-dGTPαS, 3′-O-N₃-dTTPαS, 3′-O-N₃-dUTPαS, ddCTPαSe-N₃-Bodipy-FL-510, ddUTPαSe-N₃-R6G, ddATPαSe-N₃-ROX, ddGTPαSe-N₃-Cy5, or combinations thereof.
Aspect 50. The mixture of aspect 35, wherein the Se-modified nucleotide is selected from the group consisting of dATPαSe, dCTPαSe, dGTPαSe,TTPαSe (or dTTPαSe), and dUTPαSe.
Aspect 51. The mixture of aspect 35, wherein the number of native nucleotides in the nucleotide mixture is any number disclosed herein (e.g., from 1 to 4).
Aspect 52. The mixture of aspect 35, wherein the nucleotide mixture consists of any combination of nucleotides disclosed herein (e.g., dATPαSe, dGTP, dCTP, and TTP; dATPαSe, dGTPαSe,dCTP, and TTP; dATPαSe, dGTP, dCTPαSe, and TTP; dATPαSe, dGTP, dCTP, and TTPαSe; dATPαSe, dGTPαSe,dCTPαSe, and TTPαSe; dATPαSe, dGTPαSe,dCTP, and TTPαSe; dATPαSe, dGTP, dCTPαSe, and TTPαSe; dATP, dGTPαSe,dCTPαSe, and TTPαSe, etc.).
Aspect 53. The mixture of aspect 35, wherein the nucleotide mixture comprises a combination of the Se-modified nucleotide, the S-modified nucleotide and the analogous native nucleotide.
Aspect 54. The mixture of aspect 53, wherein a molar ratio of the Se-modified nucleotide to the analogous native nucleotide is in any range disclosed herein (e.g., from 1:100 to 10:1, from 1:10 to 10:1, from 1:5 to 5:1, from 1:2 to 10:1), or analogous native nucleotide may not even be included in the reagent mixture.
Aspect 55. The process of aspect 35, wherein a concentration of each Se-modified nucleotide, each native nucleotide, and/or each combination of Se-modified nucleotide and the analogous native nucleotide, in the nucleotide mixture during the extending step, independently is in any range disclosed herein (e.g., from about 1 to about 500 µM, from about 50 µM to about 300 µM, etc.).
Aspect 56. The mixture of aspect 35, wherein the Se-modified nucleotide or the S-modified nucleotide is a mixture of diastereomers.
Aspect 57. A modified nucleotide of the formula

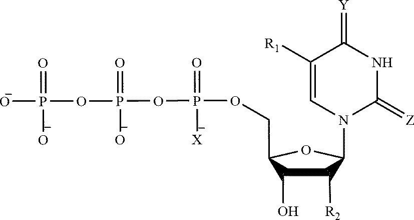

wherein:
   R₁ is CH₃ or H;
   R₂ is H or OH;
   X is Se, S, or O;
   Y is Se, S, O or NH;
   Z is Se, S, or O; and
   X, Y and Z are not each O.
Aspect 58. The modified nucleotide of aspect 57, wherein X and R₂ are O.
Aspect 59. The modified nucleotide of aspect 58, wherein Y is O.

We claim:

1. An enzymatic process for forming a nucleic acid product mixture, the process comprising:
annealing a primer or promoter sequence to a template sequence; and
extending the primer sequence or synthesizing a nucleic acid product in the presence of an extension and/or polymerization enzyme and a nucleotide mixture comprising at least one modified nucleotide to form a modified nucleic acid;
wherein an amount of nonspecific nucleic acid products in the product mixture is less than that of an otherwise identical process using an analogous native nucleotide.

2. The process of claim 1, further comprising:
isolating the modified nucleic acid; and
contacting the modified nucleic acid with an oxidative or hydrolytic solution (e.g., hydrogen peroxide) to produce an analogous native nucleic acid.

3. The process of claim 1, wherein the process is a cDNA synthesis, a PCR amplification, a rolling-circle amplification, an isothermal amplification (such as LAMP amplification), or a sequencing process.

4. The process of claim 1, wherein the native nucleic acid is a DNA.

5. The process of claim 1, wherein the native nucleotide is a human DNA nucleotide or a human RNA nucleotide.

6. The process of claim 1, wherein the native nucleotide and modified nucleotide comprise a naturally occurring base.

7. The process of claim 1, wherein the native nucleoside is a sequencing nucleotide selected from the group consisting of 3′-O-N₃-dATP, 3′-O-N₃-dCTP, 3′-O-N₃-dGTP, 3′-O-N₃-dTTP, 3′-O-N₃-dUTP, ddCTP-N₃-Bodipy-FL-510, ddUTP-N₃-R6G, ddATP-N₃-ROX, ddGTP-N₃-Cy5, or combinations thereof.

8. The process of claim 1, wherein the modified nucleotide comprises an α-phosphoselenomodified nucleotide (dNTPαSe and/or NTPαSe), an α-phosphothio-modified nucleotide (dNTPαS and/or NTPαS), or both.

9. The process of claim 1, wherein the nucleotide mixture further comprises a non-analogous native nucleotide.

10. The process of claim 1, wherein the nucleotide mixture comprises a modified nucleotide and an analogous native nucleotide, and a molar ratio of the modified nucleotide to the analogous native nucleotide is in any range from about 1:100 to about 100:1, or analogous native nucleotide may not even be included in the reagent mixture.

11. The process of claim 1, wherein the extension and/or polymerization enzyme comprises a DNA polymerase, an RNA polymerase, or a reverse transcriptase.

12. The process of claim 1, wherein an extension and/or polymerization rate of the extension and/or polymerization enzyme for the modified nucleotide is less than that for an analogous native nucleotide.

13. The process of claim 1, wherein an error rate of the extension and/or polymerization enzyme for the modified nucleotide is less than that for an analogous native nucleotide.

14. The process of claim 1, comprising repeating the annealing and extending steps for a number of cycles in a range from about 20 to about 40 or more to form an amplified nucleic acid product.

15. A reagent mixture for conducting nucleic acid extension and/or polymerization reactions, the mixture comprising:
a DNA primer sequence;
a DNA template sequence;
a DNA polymerase enzyme; and
a nucleotide mixture comprising:
a Se-modified nucleotide and a S-modified nucleotide selected from the group consisting of dATPαSe, dCTPαSe, dGTPαSe, TTPαSe (or dTTPαSe), dUTPαSe, 2-Se-TTP (or 2-Se-dTTP), 2-Se-dUTP, 2-Se-TTPαSe (or 2-Se-dTTPαSe), 2-Se-dUTPαSe, dATPαS, dCTPαS, dGTPαS, TTPαS (or dTTPαS), dUTPαS, 2-S-TTP (or 2-S-dTTP), 2-S-dUTP, 2-S-TTPαS (or 2-S-dTTPαS), 2-S-dUTPαS, or a combination thereof; and
a non-analogous native nucleotide selected from the group consisting of dATP, dGTP, dCTP, TTP, dUTP, or a combination thereof.

16. The mixture of claim 15, further comprising a reaction buffer comprising Tris-HCl, (NH₄)₂SO₄, 10 mM KCl, 2 mM MgSO₄, 0.1% Triton® X-100, or any combination thereof.

17. The mixture of claim 15, wherein:
a concentration of the template sequence is in a range from about 1 yoctoM to about 1 mM;
a concentration of the primer sequence is in a range from about 1 yoctoM to about 100 mM; and
the primer sequence comprises an Se-modified nucleotide or an S-modified nucleotide, the template sequence comprises an Se-modified nucleotide or an S-modified nucleotide, or a combination thereof.

18. A modified nucleotide selected from the group consisting of 3′-O-N₃-dATPαSe, 3′-O-N₃-dCTPαSe, 3′-O-N₃-dGTPαSe, 3′-O-N₃-dTTPαSe, 3′-O-N₃-dUTPαSe, ddCTPαSe-N₃-Bodipy-FL-510, ddUTPαSe-N₃-R6G, ddATPαSe-N₃-ROX, ddGTPαSe-N₃-Cy5, 3′-O-N₃-dATPαS, 3′-O-N₃-dCTPαS, 3′-O-N₃-dGTPαS, 3′-O-N₃-dTTPαS, 3′-O-N₃-dUTPαS, ddCTPαS-N3-Bodipy-FL-510,ddUTPαS-N₃-R6G, ddATPαS-N₃-ROX, and ddGTPαS-N₃-Cy5.

19. A reagent mixture comprising:
a primer sequence;
a template sequence;
a polymerase enzyme;
the modified nucleotide of claim 18; and
a non-analogous native nucleotide.

20. The reagent mixture of claim 19, wherein the non-analogous native nucleotide is selected from 3′-O-N₃-dATP, 3′-O-N₃-dCTP, 3′-O-N₃-dGTP, 3′-O-N₃-dTTP, ddCTP-N₃-Bodipy-FL-510, ddUTP-N₃-R6G, ddATP-N₃-ROX, and ddGTP-N₃-Cy5, or combinations thereof.

21. A reagent mixture for conducting nucleic acid extension and/or polymerization reactions, the mixture comprising:
a DNA promoter sequence;
a DNA template sequence;
a RNA polymerase enzyme; and
a nucleotide mixture comprising:
a Se-modified nucleotide and a S-modified nucleotide selected from the group consisting of ATPαSe, CTPαSe, GTPαSe, UTPαSe, rTTPαSe, 2-Se-UTP, 2-Se-rTTP, 2-Se-UTPαSe, 2-Se-rTTPαSe, ATPαS, CTPαS, GTPαS, UTPαS, rTTPαS, 2-S-UTP, 2-S-rTTP, 2-S-UTPαS, 2-S-rTTPαS, or a combination thereof; and
a non-analogous native nucleotide selected from the group consisting of ATP, CTP, GTP, UTP, rTTP, or a combination thereof.

22. A reagent mixture for conducting nucleic acid extension and/or polymerization reactions, the mixture comprising:
a primer sequence;
a RNA template sequence;
a reverse transcriptase enzyme; and
a nucleotide mixture comprising:
a Se-modified nucleotide and a S-modified nucleotide selected from the group consisting of dATPαSe, dCTPαSe, dGTPαSe, TTPαSe (or dTTPαSe), dUTPαSe, 2-Se-TTP (or 2-Se-dTTP), 2-Se-dUTP, 2-Se-TTPαSe (or 2-Se-dTTPαSe), 2-Se-dUTPαSe, dATPαS, dCTPαS, dGTPαS, TTPαS (or dTTPαS), dUTPαS, 2-S-TTP (or 2-S-dTTP), 2-S-dUTP, 2-S-TTPαS (or 2-S-dTTPαS), 2-S-dUTPαS, or a combination thereof; and
a non-analogous native nucleotide selected from the group consisting of dATP, dGTP, dCTP, TTP, dUTP, or a combination thereof.

23. A modified nucleotide of the formula 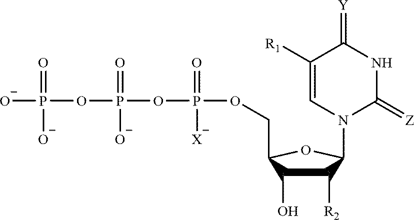
wherein:
R₁ is CH₃ or H;
R₂ is H or OH;
X is Se, S, or O;
Y is Se, S, O or NH;
Z is Se, S, or O; and
X, Y and Z are not each O.

24. The modified nucleotide of claim 23, wherein X and R₂ are O.

25. The modified nucleotide of claim 24, wherein Y is O.


SEQUENCE LISTING (34 sequences)

SEQ ID NO: 1 - DNA, 18 bp, Artificial Sequence
  Synthetic DNA sequence and provided for demonstrating the ability to reproduce DNA sequences more faithfully using PCR, by incorporating selenium-modified nucleotides into the sequence
gtcgagtcaagagcatcc

SEQ ID NO: 2 - DNA, 43 bp, Artificial Sequence
  Synthetic DNA sequence and provided for demonstrating the ability to reproduce DNA sequences more faithfully using PCR, by incorporating selenium-modified nucleotides into the sequence
cagctcagttctcgtaggttcagctaggtcagtcacatgagtc

SEQ ID NO: 3 - DNA, 43 bp, Artificial Sequence
  Synthetic DNA sequence and provided for demonstrating the ability to reproduce DNA sequences more faithfully using PCR, by incorporating selenium-modified nucleotides into the sequence
gtcgagtcaagagcatccaagtcgatccagtcagtgtactcag

SEQ ID NO: 4 - DNA, 26 bp, Artificial Sequence
  Synthetic DNA sequence and provided for demonstrating the ability to reproduce DNA sequences more faithfully using PCR, by incorporating selenium-modified nucleotides into the sequence
cagctcagttctcgtaggtcagacag

SEQ ID NO: 5 - DNA, 26 bp, Artificial Sequence
  Synthetic DNA sequence and provided for demonstrating the ability to reproduce DNA sequences more faithfully using PCR, by incorporating selenium-modified nucleotides into the sequence
cagctcagttctcgtagggtcatcta

SEQ ID NO: 6 - DNA, 26 bp, Artificial Sequence
  Synthetic DNA sequence and provided for demonstrating the ability to reproduce DNA sequences more faithfully using PCR, by incorporating selenium-modified nucleotides into the sequence
cagctcagttctcgtaggcatgtatg

SEQ ID NO: 7 - DNA, 26 bp, Artificial Sequence
  Synthetic DNA sequence and provided for demonstrating the ability to reproduce DNA sequences more faithfully using PCR, by incorporating selenium-modified nucleotides into the sequence
cagctcagttctcgtaggaatgtctg

SEQ ID NO: 8 - DNA, 99 bp, Artificial Sequence
  Synthetic DNA sequence and provided for demonstrating the ability to reproduce DNA sequences more faithfully using PCR, by incorporating selenium-modified nucleotides into the sequence
acgacgttgtaaaacgacggccagtgaattcgagctcggtacccggggatcctctagagtcgacctgcaggcatgcaagcttggcgtaatcatggtcat

SEQ ID NO: 9 - DNA, 38 bp, Artificial Sequence
  Synthetic DNA sequence and provided for demonstrating the ability to reproduce DNA sequences more faithfully using PCR, by incorporating selenium-modified nucleotides into the sequence
aatttcacacaggaaacagctatgaccatgattacgcc

SEQ ID NO: 10 - DNA, 21 bp, Artificial Sequence
  Synthetic DNA sequence and provided for demonstrating the ability to reproduce DNA sequences more faithfully using PCR, by incorporating selenium-modified nucleotides into the sequence
  modified_base (1)..(21): seleno modified nucleotides, each nucleotide in the series is a non-natural nucleotide (having an Se instead of O on the first phosphate group of each nucleotide)
dagtactagatgtgagacatc

SEQ ID NO: 11 - DNA, 67 bp, Artificial Sequence
  Synthetic DNA sequence and provided for demonstrating the ability to reproduce DNA sequences more faithfully using PCR, by incorporating selenium-modified nucleotides into the sequence
ggtcgactctagaggatccccgggtaccgagctcgaattcactggccgtcgtcgttttacaacgtcg

SEQ ID NO: 12 - DNA, 62 bp, Artificial Sequence
  Synthetic DNA sequence and provided for demonstrating the ability to reproduce DNA sequences more faithfully using PCR, by incorporating selenium-modified nucleotides into the sequence
agtcgacctgcaggcatgcaagcttggcgtaatcatggtcatagctgtttcctgtgtgaaat

SEQ ID NO: 13 - DNA, 18 bp, Artificial Sequence
  Synthetic DNA sequence and provided for demonstrating the ability to reproduce DNA sequences more faithfully using PCR, by incorporating selenium-modified nucleotides into the sequence
gtcgagtcaagagcatcc

SEQ ID NO: 14 - DNA, 26 bp, Artificial Sequence
  Synthetic DNA sequence and provided for demonstrating the ability to reproduce DNA sequences more faithfully using PCR, by incorporating selenium-modified nucleotides into the sequence
cagctcagttctcgtaggtcagacag

SEQ ID NO: 15 - DNA, 26 bp, Artificial Sequence
  Synthetic DNA sequence and provided for demonstrating the ability to reproduce DNA sequences more faithfully using PCR, by incorporating selenium-modified nucleotides into the sequence
cagctcagttctcgtagggtcatcta

SEQ ID NO: 16 - DNA, 26 bp, Artificial Sequence
  Synthetic DNA sequence and provided for demonstrating the ability to reproduce DNA sequences more faithfully using PCR, by incorporating selenium-modified nucleotides into the sequence
cagctcagttctcgtaggcatgtatg

SEQ ID NO: 17 - DNA, 26 bp, Artificial Sequence
  Synthetic DNA sequence and provided for demonstrating the ability to reproduce DNA sequences more faithfully using PCR, by incorporating selenium-modified nucleotides into the sequence
cagctcagttctcgtaggaatgtctg

SEQ ID NO: 18 - DNA, 10 bp, Artificial Sequence
  Synthetic DNA sequence and provided for demonstrating the ability to reproduce DNA sequences more faithfully using PCR, by incorporating selenium-modified nucleotides into the sequence
aacagctcct

SEQ ID NO: 19 - DNA, 26 bp, Artificial Sequence
  Synthetic DNA sequence and provided for demonstrating the ability to reproduce DNA sequences more faithfully using PCR, by incorporating selenium-modified nucleotides into the sequence
ttgtcgaggagcgggaacgagtggta

SEQ ID NO: 20 - DNA, 29 bp, Artificial Sequence
  Synthetic DNA sequence and provided for demonstrating the ability to reproduce DNA sequences more faithfully using PCR, by incorporating selenium-modified nucleotides into the sequence
gcgggaacgagtggtattgtcgatttgga

SEQ ID NO: 21 - DNA, 26 bp, Artificial Sequence
  Synthetic DNA sequence and provided for demonstrating the ability to reproduce DNA sequences more faithfully using PCR, by incorporating selenium-modified nucleotides into the sequence
gcgggaacgagtggtattgtcgagga

SEQ ID NO: 22 - DNA, 38 bp, Artificial Sequence
  Synthetic DNA sequence and provided for demonstrating the ability to reproduce DNA sequences more faithfully using PCR, by incorporating selenium-modified nucleotides into the sequence
tcaagatccgccacaacatcctgggtgctcaggtagtg

SEQ ID NO: 23 - DNA, 39 bp, Artificial Sequence
  Synthetic DNA sequence and provided for demonstrating the ability to reproduce DNA sequences more faithfully using PCR, by incorporating selenium-modified nucleotides into the sequence
cgacaagcagaagaacggcatcgcgcttctcgttggggt

SEQ ID NO: 24 - DNA, 40 bp, Artificial Sequence
  Synthetic DNA sequence and provided for demonstrating the ability to reproduce DNA sequences more faithfully using PCR, by incorporating selenium-modified nucleotides into the sequence
aagatgcgacccgattcaagcttgcagttgatcctggtgg

SEQ ID NO: 25 - DNA, 36 bp, Artificial Sequence
  Synthetic DNA sequence and provided for demonstrating the ability to reproduce DNA sequences more faithfully using PCR, by incorporating selenium-modified nucleotides into the sequence
tgcaggctgaactggtggcctccactgagcagcttg

SEQ ID NO: 26 - DNA, 45 bp, Artificial Sequence
  Synthetic DNA sequence and provided for demonstrating the ability to reproduce DNA sequences more faithfully using PCR, by incorporating selenium-modified nucleotides into the sequence
ttggagaggtcatcaggaagggttggctacttccagttttactcc

SEQ ID NO: 27 - DNA, 36 bp, Artificial Sequence; identical to SEQ ID NO: 25
  Synthetic DNA sequence and provided for demonstrating the ability to reproduce DNA sequences more faithfully using PCR, by incorporating selenium-modified nucleotides into the sequence

SEQ ID NO: 28 - DNA, 43 bp, Artificial Sequence
  Synthetic DNA sequence and provided for demonstrating the ability to reproduce DNA sequences more faithfully using PCR, by incorporating selenium-modified nucleotides into the sequence
gcaatgagctcatggcttcctttttaagaagaggttttctggg

SEQ ID NO: 29 - DNA, 10 bp, Artificial Sequence
  Synthetic DNA sequence and provided for demonstrating the ability to reproduce DNA sequences more faithfully using PCR, by incorporating selenium-modified nucleotides into the sequence
cgtcttggcc

SEQ ID NO: 30 - DNA, 30 bp, Artificial Sequence
  Synthetic DNA sequence and provided for demonstrating the ability to reproduce DNA sequences more faithfully using PCR, by incorporating selenium-modified nucleotides into the sequence
gcagaaccggatgtcgtccgtcggcgcttg

SEQ ID NO: 31 - DNA, 23 bp, Artificial Sequence
  Seq32
gcagaaccggtcgcttcctcttc

SEQ ID NO: 32 - DNA, 24 bp, Artificial Sequence
  Synthetic DNA sequence and provided for demonstrating the ability to reproduce DNA sequences more faithfully using PCR, by incorporating selenium-modified nucleotides into the sequence
gcagaaccggtcggcttcctcttc

SEQ ID NO: 33 - DNA, 25 bp, Artificial Sequence
  Synthetic DNA sequence and provided for demonstrating the ability to reproduce DNA sequences more faithfully using PCR, by incorporating selenium-modified nucleotides into the sequence
gcagaaccggtcgggcttcctcttc

SEQ ID NO: 34 - DNA, 30 bp, Artificial Sequence; identical to SEQ ID NO: 30
  Synthetic DNA sequence and provided for demonstrating the ability to reproduce DNA sequences more faithfully using PCR, by incorporating selenium-modified nucleotides into the sequence